SEQUENCE LISTING (10 sequences)

SEQ ID NO: 1 - RNA, 21 nt, synthetic construct
  misc_feature 1..21: note = Description of sequence: siRNA
  source 1..21: mol_type = other RNA
cacggaacatgttacttcatt

SEQ ID NO: 2 - RNA, 21 nt, synthetic construct
  misc_feature 1..21: note = Description of sequence: siRNA
  source 1..21: mol_type = other RNA
tgaagtaacatgttccgtgtt

SEQ ID NO: 3 - RNA, 21 nt, synthetic construct
  misc_feature 1..21: note = Description of sequence: siRNA
  source 1..21: mol_type = other RNA
ggatccaatgtgtcaatgatt

SEQ ID NO: 4 - RNA, 21 nt, synthetic construct
  misc_feature 1..21: note = Description of sequence: siRNA
  source 1..21: mol_type = other RNA
tcattgacacattggatcctt

SEQ ID NO: 5 - RNA, 21 nt, synthetic construct
  misc_feature 1..21: note = Description of sequence: siRNA
  source 1..21: mol_type = other RNA
gatccaatgtgtcaatgaatt

SEQ ID NO: 6 - RNA, 21 nt, synthetic construct
  misc_feature 1..21: note = Description of sequence: siRNA
  source 1..21: mol_type = other RNA
ttcattgacacattggatctt

SEQ ID NO: 7 - RNA, 21 nt, synthetic construct
  misc_feature 1..21: note = Description of sequence: shRNA
  source 1..21: mol_type = other RNA
gcagggtcccagtccaattaa

SEQ ID NO: 8 - DNA, 2630 bp, Homo sapiens
  misc_feature 1..2630: note = Description of sequence: Homo sapiens cleavage stimulation factor subunit 2 (CSTF2), transcript variant 1, mRNA (NM 001306206.2)
  source 1..2630: mol_type = genomic DNA
gcaccggaagccgactcaacagagctatggcgggtttgactgtgagagacccagcggtggatcgttctctacgttctgtgttcgtggggaacattccttatgaagctactgaagagcagttgaaggacatcttttctgaggttggacctgttgttagtttcagattggtatacgatagagagacaggaaagccaaagggttatggcttctgtgaataccaagaccaagagacagcacttagtgccatgcggaacctgaatgggcgcgaattcagtgggagagcacttcgagtggacaatgctgccagtgaaaagaacaaagaagagctgaagagccttggcactggtgcccctgtcattgagtcaccttatggagagaccatcagtcctgaggatgcccctgagtccattagcaaagcagttgccagccttccaccagagcagatgtttgagctgatgaaacaaatgaagctctgtgtccagaatagtccccaggaggcacggaacatgttacttcagaaccctcaactggcttatgctttgctgcaagcacaggtagtgatgagaattgtggatccggaaattgccctgaaaattctgcatcgccagacaaatatcccaacgctgattgcaggcaaccctcagccagtccatggtgctgggcctggctcaggatccaatgtgtcaatgaaccagcagaatcctcaggcccctcaggcccagtctttgggtggaatgcatgtcaatggcgcacctcctctgatgcaagcttctatgcagggtggagttccagcaccagggcaaatgccagctgctgtcacaggacctggccctggttccttagctcctggaggaggaatgcaggctcaggttggaatgccaggaagtggaccagtgtccatggaacgggggcaaggaaccctacagcactcgcccgtgggacccgccgggcctgcatcaattgagcgagttcaagtgccgatgcaagaccccagagcagctatgcagcggggatccttgcctgcgaatgtcccaacccctcgaggcttgttaggagatgctccgaatgatccacggggaggcactttactttctgtaactggagaggtagagcctagaggttacttgggaccacctcatcagggtccacccatgcaccatgtccctggccatgagagccgaggaccacccccacatgaactgaggggagggccattacccgagcccagacctctaatggcagaaccaagaggacccatgctagatcagaggggtccacccttggatggcagaggtggaagggatccccgaggaatagatgcacgagggatggaggcccgagccatggaggcaagagggttagatgccagaggattagaggcccgtgcaatggaggcccgtgcgatggaagctcgtgcaatggaggcccgagcgatggaggcccgtgcaatggaagtccgagggatggaggccagaggcatggataccagaggcccagtgcctggccccagaggacctatacctagtggaatgcagggtcccagtccaattaacatgggggcggttgtcccccagggatccagacaggtcccagtcatgcagggaacaggaatgcaaggagcaagtatacagggtggaagccagcctggcggctttagtcccgggcagaaccaagtcactccacaggatcatgagaaggctgctttgattatgcaggttctacaactgactgcagaccagattgccatgttgcctcctgagcaaaggcagagtatcctgattttaaaggaacaaatacagaaatccactggagcaccttgataggttttcaaaaatacctggcaagaaatctggaaattctataattttgttgaaatattgaaaaaagatgacctgcatcctaacccttgaatgactcaaatcagtgccaggtggaggactcccatcaccttctctcagaacaaaatcacttcattttattgtcttagtttgtatattttctgtgacttgaaataaactttgaacacaattttagtacactgcaaattgatacacatgacctttttgcttttaaaaagcttaacactaagggtgaaaccttagatgtgttttctacccttactgcagtattgtactttaacatattgccatagttttgtttttaagttaaccacttattcttcttgatgatatttgagaatttctcagaagcaatgttttctggaacttggtttttgtttagtttgcttccagtaagaattattataaggctttgccctctgaaaatattctagcacttcggagtcttctaggagaaaatatcacatgcttagagctataaaatggtcagaaggctaacagagcaaagagcctagcacagctgtgtgtttagtttagaaaaatccaaatatcaaaatgcatttgtttcaatagcaggtctgaacatgtataattcagagttaagacaaaaattgttttagcactggtttgagtgtgtcagcaacacaagagactgttgacatttacttgtgtgttcttaaacttatcagtctaggcagtcaactttgaaagtttaaaaagccactgatttttcacatgctcaagatgaaataaaaacagaaatcaaatgaa

SEQ ID NO: 9 - DNA, 2570 bp, Homo sapiens
  misc_feature 1..2570: note = Description of sequence: Homo sapiens cleavage stimulation factor subunit 2 (CSTF2), transcript variant 2, mRNA (NM001325.3)
  source 1..2570: mol_type = genomic DNA
gcaccggaagccgactcaacagagctatggcgggtttgactgtgagagacccagcggtggatcgttctctacgttctgtgttcgtggggaacattccttatgaagctactgaagagcagttgaaggacatcttttctgaggttggacctgttgttagtttcagattggtatacgatagagagacaggaaagccaaagggttatggcttctgtgaataccaagaccaagagacagcacttagtgccatgcggaacctgaatgggcgcgaattcagtgggagagcacttcgagtggacaatgctgccagtgaaaagaacaaagaagagctgaagagccttggcactggtgcccctgtcattgagtcaccttatggagagaccatcagtcctgaggatgcccctgagtccattagcaaagcagttgccagccttccaccagagcagatgtttgagctgatgaaacaaatgaagctctgtgtccagaatagtccccaggaggcacggaacatgttacttcagaaccctcaactggcttatgctttgctgcaagcacaggtagtgatgagaattgtggatccggaaattgccctgaaaattctgcatcgccagacaaatatcccaacgctgattgcaggcaaccctcagccagtccatggtgctgggcctggctcaggatccaatgtgtcaatgaaccagcagaatcctcaggcccctcaggcccagtctttgggtggaatgcatgtcaatggcgcacctcctctgatgcaagcttctatgcagggtggagttccagcaccagggcaaatgccagctgctgtcacaggacctggccctggttccttagctcctggaggaggaatgcaggctcaggttggaatgccaggaagtggaccagtgtccatggaacgggggcaagtgccgatgcaagaccccagagcagctatgcagcggggatccttgcctgcgaatgtcccaacccctcgaggcttgttaggagatgctccgaatgatccacggggaggcactttactttctgtaactggagaggtagagcctagaggttacttgggaccacctcatcagggtccacccatgcaccatgtccctggccatgagagccgaggaccacccccacatgaactgaggggagggccattacccgagcccagacctctaatggcagaaccaagaggacccatgctagatcagaggggtccacccttggatggcagaggtggaagggatccccgaggaatagatgcacgagggatggaggcccgagccatggaggcaagagggttagatgccagaggattagaggcccgtgcaatggaggcccgtgcgatggaagctcgtgcaatggaggcccgagcgatggaggcccgtgcaatggaagtccgagggatggaggccagaggcatggataccagaggcccagtgcctggccccagaggacctatacctagtggaatgcagggtcccagtccaattaacatgggggcggttgtcccccagggatccagacaggtcccagtcatgcagggaacaggaatgcaaggagcaagtatacagggtggaagccagcctggcggctttagtcccgggcagaaccaagtcactccacaggatcatgagaaggctgctttgattatgcaggttctacaactgactgcagaccagattgccatgttgcctcctgagcaaaggcagagtatcctgattttaaaggaacaaatacagaaatccactggagcaccttgataggttttcaaaaatacctggcaagaaatctggaaattctataattttgttgaaatattgaaaaaagatgacctgcatcctaacccttgaatgactcaaatcagtgccaggtggaggactcccatcaccttctctcagaacaaaatcacttcattttattgtcttagtttgtatattttctgtgacttgaaataaactttgaacacaattttagtacactgcaaattgatacacatgacctttttgcttttaaaaagcttaacactaagggtgaaaccttagatgtgttttctacccttactgcagtattgtactttaacatattgccatagttttgtttttaagttaaccacttattcttcttgatgatatttgagaatttctcagaagcaatgttttctggaacttggtttttgtttagtttgcttccagtaagaattattataaggctttgccctctgaaaatattctagcacttcggagtcttctaggagaaaatatcacatgcttagagctataaaatggtcagaaggctaacagagcaaagagcctagcacagctgtgtgtttagtttagaaaaatccaaatatcaaaatgcatttgtttcaatagcaggtctgaacatgtataattcagagttaagacaaaaattgttttagcactggtttgagtgtgtcagcaacacaagagactgttgacatttacttgtgtgttcttaaacttatcagtctaggcagtcaactttgaaagtttaaaaagccactgatttttcacatgctcaagatgaaataaaaacagaaatcaaatgaa

SEQ ID NO: 10 - DNA, 2519 bp, Homo sapiens
  misc_feature 1..2519: note = Description of sequence: Homo sapiens cleavage stimulation factor subunit 2 (CSTF2), transcript variant 3, mRNA (NM001306209.2)
  source 1..2519: mol_type = genomic DNA
gcaccggaagccgactcaacagagctatggcgggtttgactgtgagagacccagcggtggatcgttctctacgttctgtgttcgtggggaacattccttatgaagctactgaagagcagttgaaggacatcttttctgaggttggacctgttgttagtttcagattggtatacgatagagagacaggaaagccaaagggttatggcttctgtgaataccaagaccaagagacagcacttagtgccatgcggaacctgaatgggcgcgaattcagtgggagagcacttcgagtggacaatgctgccagtgaaaagaacaaagaagagctgaagagccttggcactggtgcccctgtcattgagtcaccttatggagagaccatcagtcctgaggatgcccctgagtccattagcaaagcagttgccagccttccaccagagcagatgtttgagctgatgaaacaaatgaagctctgtgtccagaatagtccccaggaggcacggaacatgttacttcagaaccctcaactggcttatgctttgctgcaagcacaggtagtgatgagaattgtggatccggaaattgccctgaaaattctgcatcgccagacaaatatcccaacgctgattgcaggcaaccctcagccagtccatggtgctgggcctggctcaggatccaatgtgtcaatgaaccagcagaatcctcaggcccctcaggcccagtctttgggtggagttccagcaccagggcaaatgccagctgctgtcacaggacctggccctggttccttagctcctggaggaggaatgcaggctcaggttggaatgccaggaagtggaccagtgtccatggaacgggggcaagtgccgatgcaagaccccagagcagctatgcagcggggatccttgcctgcgaatgtcccaacccctcgaggcttgttaggagatgctccgaatgatccacggggaggcactttactttctgtaactggagaggtagagcctagaggttacttgggaccacctcatcagggtccacccatgcaccatgtccctggccatgagagccgaggaccacccccacatgaactgaggggagggccattacccgagcccagacctctaatggcagaaccaagaggacccatgctagatcagaggggtccacccttggatggcagaggtggaagggatccccgaggaatagatgcacgagggatggaggcccgagccatggaggcaagagggttagatgccagaggattagaggcccgtgcaatggaggcccgtgcgatggaagctcgtgcaatggaggcccgagcgatggaggcccgtgcaatggaagtccgagggatggaggccagaggcatggataccagaggcccagtgcctggccccagaggacctatacctagtggaatgcagggtcccagtccaattaacatgggggcggttgtcccccagggatccagacaggtcccagtcatgcagggaacaggaatgcaaggagcaagtatacagggtggaagccagcctggcggctttagtcccgggcagaaccaagtcactccacaggatcatgagaaggctgctttgattatgcaggttctacaactgactgcagaccagattgccatgttgcctcctgagcaaaggcagagtatcctgattttaaaggaacaaatacagaaatccactggagcaccttgataggttttcaaaaatacctggcaagaaatctggaaattctataattttgttgaaatattgaaaaaagatgacctgcatcctaacccttgaatgactcaaatcagtgccaggtggaggactcccatcaccttctctcagaacaaaatcacttcattttattgtcttagtttgtatattttctgtgacttgaaataaactttgaacacaattttagtacactgcaaattgatacacatgacctttttgcttttaaaaagcttaacactaagggtgaaaccttagatgtgttttctacccttactgcagtattgtactttaacatattgccatagttttgtttttaagttaaccacttattcttcttgatgatatttgagaatttctcagaagcaatgttttctggaacttggtttttgtttagtttgcttccagtaagaattattataaggctttgccctctgaaaatattctagcacttcggagtcttctaggagaaaatatcacatgcttagagctataaaatggtcagaaggctaacagagcaaagagcctagcacagctgtgtgtttagtttagaaaaatccaaatatcaaaatgcatttgtttcaatagcaggtctgaacatgtataattcagagttaagacaaaaattgttttagcactggtttgagtgtgtcagcaacacaagagactgttgacatttacttgtgtgttcttaaacttatcagtctaggcagtcaactttgaaagtttaaaaagccactgatttttcacatgctcaagatgaaataaaaacagaaatcaaatgaa

(19) United States
(12) Patent Application Publication
(10) Pub. No.: US 2026/0337608 A1
(43) Pub. Date: Oct. 29, 2026

(54) MODULATORS OF ALTERNATIVE POLYADENYLATION AND METHODS OF USE THEREOF

(71) Applicant: THE METHODIST HOSPITAL, Houston, TX (US)

(72) Inventors: Rajarajan AMIRTHALINGAM THANDAVARAYAN, Houston, TX (US); Rahul Dev NEUPANE, Houston, TX (US); Ashrith GUHA, Bellaire, TX (US)

(21) Appl. No.: 19/150,772

(22) PCT Filed: Jan. 26, 2024
(86) PCT No.: PCT/US2024/013140

§ 371 Date: Jul. 24, 2025

Related U.S. Application Data
(60) Provisional application No. 63/481,810, filed on Jan. 27, 2023.

(51) Int. Cl. C12N 15/113; A61P 9/04

(52) U.S. Cl. CPC C12N 15/113; A61P 9/04; C12N 2310/14; C12N 2310/531

(57) ABSTRACT

The present disclosure relates to compositions and methods of treating, preventing, or diagnosing cardiac fibrosis and related diseases by modulating alternative polyadenylation.

### CROSS-REFERENCE TO RELATED APPLICATIONS

This PCT application claims priority to, and the benefit of, U.S. Provisional Patent Application No. 63/481,810, filed Jan. 27, 2023, which is incorporated by reference herein in its entirety.

### REFERENCE TO SEQUENCE LISTING

The sequence listing submitted on Jan. 26, 2024, as an .XML file entitled “10063-082WO1_ST26.xml” created on Jan. 24, 2024, and having a file size of 21,694 bytes is hereby incorporated by reference pursuant to 37 C.F.R. § 1.52(e)(5).

### FIELD

The present disclosure relates to compositions and methods of treating, preventing, or diagnosing cardiac fibrosis and related diseases by modulating alternative polyadenylation.

### BACKGROUND

Cleavage and polyadenylation of mRNA was thought to be a constitutive process, but recent discoveries have shown that not only it is a regulated process, but many mRNAs also contain multiple polyadenylation sites that allow for major changes in 3′UTR length. Upregulation of polyadenylation protein complexes can cause global 3′UTRs shortening, which can increase transcript stability and protein expression of genes, thereby allowing transcripts to escape mRNA regulation resulting in heightened translation of affected transcripts. Although alternative polyadenylation (APA) drives tumorigenic properties in cancer, promotes lung disease and modulates HF, its role as a mediator of cardiac fibrosis is not well understood. In addition, there are few therapeutic options accompanied with poor prognosis for heart failure associated with cardiac fibrosis.

Given limitations of available therapeutics to treat or prevent cardiac fibrosis and related diseases, there is a need to address the aforementioned problems mentioned above by developing methods to reduce or prevent dysfunction associated with cardiac fibrosis. The methods disclosed herein address these and other needs.

### SUMMARY

The present disclosure provides methods of treating, preventing, decreasing, reducing, and/or ameliorating a cardiovascular disease including, but not limited to cardiac fibrosis and heart failure, in a subject using an inhibitor of CstF64 or an inhibitor cocktail of CstF64. The present disclosure also provides compositions comprising an inhibitor of CstF64 or an inhibitor cocktail of CstF64 and a pharmaceutically acceptable carrier.

In one aspect, disclosed herein is a method of treating or preventing cardiac fibrosis, comprising administering to a subject in need thereof a therapeutically effective amount of a composition comprising an inhibitor of CstF64.

In one aspect, disclosed herein is a method of treating or preventing heart failure, comprising administering to a subject in need thereof a therapeutically effective amount of a composition comprising an inhibitor of CstF64.

In some embodiments, the inhibitor of CstF64 is selected from a nucleic acid or a small molecule. In some embodiments, the inhibitor of CstF64 comprises at least one siRNA. In some embodiments, the inhibitor of CstF64 comprises one, two, or three siRNAs.

In some embodiments, the siRNAs comprises SEQ ID NO: 1, SEQ ID NO: 2, SEQ ID NO: 3, SEQ ID NO: 4, SEQ ID NO: 5, SEQ ID NO: 6, or any combination thereof. In some embodiments, the siRNA comprises SEQ ID NO: 1. In some embodiments, the siRNA comprises SEQ ID NO: 2. In some embodiments, the siRNA comprises SEQ ID NO: 3. In some embodiments, the siRNA comprises SEQ ID NO: 4. In some embodiments, the siRNA comprises SEQ ID NO: 5. In some embodiments, the siRNA comprises SEQ ID NO: 6.

In some embodiments, the inhibitor of CstF64 comprises a shRNA. In some embodiments, the shRNA comprises SEQ ID NO: 7.

In one aspect, disclosed herein is a method of diagnosing a subject with heart failure or cardiac fibrosis, comprising obtaining a biological sample from a subject, determining an expression level of CstF64, and determining the subject has heart failure if the expression level of CstF64 is increased in the subject in comparison to a control sample. In some embodiments, the expression level of CstF64 is measured by mRNA levels. In some embodiments, the expression level of CstF64 is measured by protein levels.

In one aspect, disclosed herein is a composition comprising an inhibitor cocktail and a pharmaceutically acceptable carrier, wherein the inhibitor cocktail comprises at least one inhibitor of CstF64 selected from SEQ ID NO: 1, SEQ ID NO: 2, SEQ ID NO: 3, SEQ ID NO: 4, SEQ ID NO: 5, SEQ ID NO: 6, or SEQ ID NO: 7.

In some embodiments, the inhibitor cocktail comprises one, two, or three inhibitors of CstF64. In some embodiments, the inhibitor cocktail comprises an siRNA or an shRNA. In some embodiments, the inhibitor cocktail comprises SEQ ID NO: 1, SEQ ID NO: 2, SEQ ID NO: 3, SEQ ID NO: 4, SEQ ID NO: 5, SEQ ID NO: 6, or any combination thereof.

In some embodiments, the inhibitor cocktail comprises SEQ ID NO: 1. In some embodiments, the inhibitor cocktail comprises SEQ ID NO: 2. In some embodiments, the inhibitor cocktail comprises SEQ ID NO: 3. In some embodiments, the inhibitor cocktail comprises SEQ ID NO: 4. In some embodiments, the inhibitor cocktail comprises SEQ ID NO: 5. In some embodiments, the inhibitor cocktail comprises SEQ ID NO: 6. In some embodiments, the inhibitor cocktail comprises SEQ ID NO: 7.

### BRIEF DESCRIPTION OF FIGURES

The accompanying figures, which are incorporated in and constitute a part of this specification, illustrate several aspects described below.

FIG. 1 shows the diagram of alternative cleavage and polyadenylation (APA) in fibrotic mRNAs. In fibrotic environments, a switch from distal polyadenylation (dPAS) to proximal poly-adenylation (pPAS) occurs due to upregulation of a key component of the cleavage/polyadenylation machinery, CSTF2 (CFTF2 is alternatively referred to herein as CstF64). This results in mRNAs with shortened 3′UTRs that can no longer be regulated by microRNAs, which leads to enhanced pro-fibrotic gene activation and protein production.

FIGS. 2A and 2B show the PAC-seq of human heart tissues from HF. FIG. 2A shows the representative scatter plot of poly A Index values. FIG. 2B shows the UCSC genome browser view of a 3′ UTR shortened gene showing usage of additional proximal APA site in the HF (bottom, red peaks) versus healthy heart (top, orange peaks).

FIGS. 3A, 3B, 3C, and 3D show that upregulation of APA regulators cause 3′UTR shortening of fibrosis related genes in human HF tissues. FIG. 3A shows the representative photomicrographs of control and HF (Pre-LVAD and Post-LVAD) tissue cross-sections stained with Masson's trichrome (Left). Scale bar, 20 μm. Bar plot showing the area of fibrosis represented as the percentage of cardiac fibrosis (Right). FIG. 3B shows the western blot showing expression of CSTF2, FN, α-SMA, and GAPDH proteins in control and HF (Pre-LVAD and Post-LVAD). Plot showing dPAS usage in Figure HF vs control and FIG. 3D Post-LAVD vs Pre-LVAD tissues by qPCR. Data are presented as mean±SEM. n=3, *p<0.05; **p<0.01; ***p<0.001.

FIGS. 4A, 4B, 4C, 4D, 4E, and 4F show the upregulation of APA regulators cause 3′UTR shortening of fibrosis related genes in non-ischemic mouse model of HF. FIG. 4A shows the experimental model where C57/BL6 mice underwent HF induction over a 5-week period. Each group had 5 mice and were sacrificed at the end of 5 weeks. L-NAME and NaCl were administered in drinking water and angiotensin II delivered by osmotic mini-pumps. L-NAME, NG-nitro-L-Arginine Methyl Ester; NaCl, Sodium Chloride; ATII, Angiotensin II. FIG. 4B shows the ejection fraction in control and HF mice. FIG. 4C shows the representative photomicrographs of control and heart failure mice tissue sections stained with Masson's trichrome (Left). Scale bar, 50 μm. Bar plot showing the area of fibrosis represented as the percentage of cardiac fibrosis (Right). FIG. 4D shows the bar plot showing CSTF2 expression in control and HF mice by qPCR. FIG. 4E shows the plot showing dPAS usage in HF vs control mice tissues by qPCR. FIG. 4F shows the immunohistochemistry showing CSTF2 expression and dual immunostaining showing CSTF2 (red) and α-SMA (green) expression in control and HF mice (Left). Scale bar, 20 μm. Number of CSTF2 positive cells (Top) and CSTF2 and α-SMA dual positive cells (Bottom) in control and HF mice sections (Right). n=3, *p<0.05; **p<0.01; ***p<0.001.

FIGS. 5A, 5B, 5C, 5D, and 5E show the CSTF2 target genes involved in fibrotic pathways in human cardiac fibroblast. FIG. 5A shows the immunofluorescence staining of CSTF2 in HF fibroblasts. FIG. 5B shows the quantification of CSTF2 expression in the control and HF fibroblasts. FIG. 5C shows the plot showing mRNA expression of α-SMA, TGF-β1, COL1A, FN1, and TGRβR1 in HF fibroblasts. FIG. 5D shows the dPAS usage in HF fibroblasts. FIG. 5E shows the immunofluorescence staining of COL1A, FN1, TGF-β1, and α-SMA proteins in HF fibroblasts. Scale bar, 150 μm. Data are presented as mean±SEM. n=3, *p<0.05; **p<0.01; ***p<0.001.

FIGS. 6A and 6B show the CSTF2 knockdown lengthens the 3′UTR of profibrotic genes in TGFβ1 treated human cardiac fibroblasts. FIG. 6A shows the CSTF2, COL1A, FN1, and α-SMA expression in control, TGFβ1 treated fibroblasts with control siRNA, and TGFβ1 treated fibroblasts with CSTF2 siRNA. Scale bars, 150 μm. FIG. 6B shows the plot showing dPAS usage after TGFβ1 treatment (Top) and treatment with CSTF2 siRNA (Bottom). Data are presented as mean±SEM. Student's t-test was used to analyze the data; n=3, *p<0.05 and **p<0.01.

FIGS. 7A, 7B, 7C, 7D, and 7E show the overexpression of CSTF2 leads to 3′UTR shortening of fibrotic genes in mouse cardiac fibroblasts. FIG. 7A shows the four- to six-week-old C57/BL6 mice is administrated AAV9-hTCF21-GFP or AAV9-TCF21-CSTF2-GFP to induce CSTF2 expression in cardiac fibroblasts. n=10 biological replications. FIG. 7B shows the human control cardiac fibroblasts transfected with AAV9-control-GFP and AAV9-CSTF2-GFP. Scale bar, 150 μm. FIG. 7C shows the quantification of CSTF2 mRNA expression after CSTF2 overexpression. FIG. 7D shows the plot showing dPAS usage in control fibroblasts treated with Ad-CSTF2. FIG. 7E shows the immunofluorescence staining of COL1A, FN1, TGFβ1, and α-SMA proteins in control fibroblasts treated with Ad-CSTF2. Scale bar, 150 μm. Data are presented as mean±SEM. Student's t-test was used to analyze the data; n=3, *p<0.05; **p<0.01; ***p<0.001.

FIGS. 8A, 8B, 8C, 8D, 8E, 8F, and 8G show the miR targeting of CSTF2. FIG. 8A shows the schematic representation of human CSTF2 3′UTR binding sites by five predicted by TargetScanHuman 7.2. FIGS. 8B, 8C, 8D, and 8E show the bar plot showing expression of miR-124 in (FIG. 8B) HF and control tissues, (FIG. 8C) HF and control fibroblasts, (FIG. 8D) control and TGFβ1 treated fibroblasts, and (FIG. 8E) HF and control mice tissues. FIGS. 8F and 8G show the bar plot showing the expression of (FIG. 8F) miR-124 and (FIG. 8G) CSTF2 in fibroblasts treated with miR-124 inhibitor by qPCR. n=3, *p<0.05; **p<0.01; ***p<0.001.

FIG. 9 shows that upregulation of CstF64 leads to 3′UTR shortening.

FIG. 10 shows the CstF64 and COL1A expression in control and TGFβ1 treated mouse cardiac fibroblasts. Scale bars, 150 μm.

FIGS. 11A, 11B, 11C, 11D, 11E, and 11F show CstF64 expression and 3′UTR changes in profibrotic genes in the left ventricle (LV) tissues of heart failure (HF) patients. FIG. 11A shows the western blot showing expression of CstF64 and internal control GAPDH proteins in LV tissues of healthy and HF patients. FIG. 11B shows the densitometry analysis of the expression of CstF64 from western blot normalized to GAPDH. FIG. 11C shows the representative photomicrographs of healthy and HF LV tissue cross-section stained with Masson's Trichrome. Scale bar, 50 μm. FIG. 11D shows the plot showing mRNA expression of COL1A, FN1, and TGFβR1 in HF and healthy LV tissues by RT-qPCR. FIG. 11E shows an illustration depicting primers used to detect total (P1) and long variants (P2) transcript. FIG. 11F shows the plot showing significant shortening of COL1A and FN1 in HF LV tissues compared to healthy donors by dPAS using RT-qPCR. Data are presented as mean±SEM. Student's t-test was used to analyze the data; n=3, *p<0.05 and **p<0.01.

FIGS. 12A, 12B, 12C, 12D, and 12E show the CstF64 expression and 3′UTR changes in profibrotic genes within isolated fibroblasts from the HF patients' LV. FIG. 12A shows the immunofluorescence staining of CstF64 expression in isolated fibroblasts from LV of HF patients. Scale bar, 150 μm. FIG. 12B shows the bar plot showing quantification of CstF64 expression from immunofluorescence staining normalized to DAPI in the control and HF fibroblasts. FIG. 12C shows the plot showing mRNA expression of α-SMA, TGF-β1, COL1A, FN1, and TGRβR1 in heart failure and control fibroblast by RT-qPCR. FIG. 12D shows the plot showing significant shortening of COL1A, FN1, and TGFβR1 in HF fibroblasts compared to control by dPAS usage RT-qPCR. FIG. 12E shows the immunofluorescence staining of COL1A, FN1, TGF-β1, and α-SMA proteins in isolated fibroblasts from LV of HF patients. Scale bar, 150 μm. Data are presented as mean±SEM. Student's t-test was used to analyze the data; n=3, *p<0.05; **p<0.01; ***p<0.001.

FIGS. 13A and 13B show the CstF64 knockdown attenuates fibrotic protein expression in HF fibroblasts. FIG. 13A shows the immunofluorescence staining of CstF64, COL1A, TGF-β1, and FN1 in an HF fibroblast 72 hours post-transfection with control and CstF64 siRNA. Scale bar, 150 μm. FIG. 13B shows the bar plots showing quantification of CstF64, TGF-β1, COL1A, and FN1 protein expression normalized to DAPI corresponding to the immunofluorescence staining in FIG. 13A. Data are represented as mean±SEM. Student's t-test was used to analyze the data; n=3, *p<0.05; **p<0.01; ***p<0.001.

FIGS. 14A, 14B, and 14C shows the CstF64 knockdown promotes the 3′UTR lengthening of profibrotic genes expression in HF fibroblasts. FIG. 14A shows the bar plot showing relative mRNA expression of COL1A, FN1, TGRβR1, TGF-β1, and α-SMA in an HF fibroblast 72 hours post-transfection with CstF64 and control siRNA by RT-qPCR. FIG. 14B shows the plot showing significant lengthening of COL1A, FN1, and TGFβR1 in HF fibroblasts 72 hours post-transfection with CstF64 siRNA compared to control siRNA by dPAS using RT-qPCR. Data are represented as mean±SEM. Student's t-test was used to analyze the data; n=3, *p<0.05; **p<0.01. FIG. 14C shows the model illustrating the effect of CstF64 upregulation on cardiac fibrosis.

FIGS. 15A, 15B, 15C, 15D, 15E, and 15F show the gene expression and alternative polyadenylation (APA) in heart failure (HF) mouse heart tissues with control shRNA and CstF64 shRNA. FIG. 15A shows the depiction of the experimental model. FIG. 15B shows the plot showing relative CstF64 mRNA expression in HF mice after AAV9 treatments. FIGS. 15C, 15D, 15E, and 15F show the dPAS usage (FIG. 15C) and gene expression change (FIGS. 15D, 15E, and 15F) of profibrotic transcripts in AAV9-treated HF mice heart tissues by qPCR. Data are presented as mean±SEM. n=3, *p<0.05; ***p<0.001.

FIGS. 16A and 16B show the fibrosis in HF mouse heart tissues with control shRNA and CstF64 shRNA. FIG. 16A shows the Masson's Trichrome staining shows reduction of fibrosis with AAV-CstF64 shRNA treatment. Scale bar, 20 μm. FIG. 16B shows the quantification of Masson's Trichrome staining from FIG. 16A.

FIGS. 17A and 17B show the hypertrophy in HF mouse heart tissues with control shRNA and CstF64 shRNA. FIG. 17A shows the hematoxylin and eosin (H&E) staining performed in HF mice treated with AAV-con shRNA or AAV-CstF64 shRNA. Scale bar, 20 μm. FIG. 17B shows the box plot showing the cardiomyocyte diameter measured from FIG. 17A.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### TERMINOLOGY

Unless defined otherwise, all technical and scientific terms used herein have the same meaning as commonly understood to one of ordinary skill in the art to which this disclosure belongs. The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The term “mRNA” refers to messenger ribonucleic acid, or single stranded molecule of RNA that corresponds to the genetic sequence of a gene and is translated by a ribosome in the process of synthesizing a protein. mRNA is created during the process of transcription, where a gene is converted into a primary transcript mRNA (or pre-mRNA). The primary transcript is further processed through RNA splicing to only contain regions that will encode protein. mRNA can also be targeted for epigenetic modifications, such as methylation, to impact mRNA translation, nuclear retention, nuclear export, processing, and splicing.

A “shRNA” also termed a “short hairpin RNA” or “small hairpin RNA” is an artificial RNA molecule with a tight hairpin turn that is used to silence target gene expression. The turn within the artificial RNA molecule prevents or silence gene expression of the desired or target gene.

As used herein, an “siRNA”, also referred to as silencing RNA or short interfering RNA, are double-stranded, non-coding RNA molecules that interfere with the expression of specific genes with complementary nucleotide sequences degrading mRNA after transcription, thus preventing translation.

“Expression” as used herein refers to the process by which information from a gene is used in the synthesis of a functional gene product that enables it to produce a peptide/protein end product, and ultimately affect a phenotype, as the final effect. Used herein, an “expression level” refers to the quantification of a functional gene product, either mRNA or protein, used to determine how much a gene is being expressed or transcribed.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is less than 100%. As used herein, an inhibitor or antagonist includes but is not limited to small molecules, nucleic acids, peptides, antibodies, or compounds.

The term “administer”, “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset, during early onset, or after an established development of disease. Prophylactic administration can occur for several days to years prior to the manifestation of symptoms of an infection.

The term “diagnose”, “diagnosing”, “diagnosed”, or derivatives thereof refer to the process of identifying the nature and cause of a certain phenomenon, including, but not limited to diseases, disorders, malignancies, and conditions. Specifically, diagnosing is an attempt to classify and determine which diseases, disorders, malignancies, or conditions are causing the symptoms and signs showing in a subject.

### Inhibitor Compositions

In one aspect, disclosed herein is a composition comprising an inhibitor cocktail and a pharmaceutically acceptable carrier, wherein the inhibitor cocktail comprises at least one inhibitor of CstF64 selected from SEQ ID NO: 1, SEQ ID NO: 2, SEQ ID NO: 3, SEQ ID NO: 4, SEQ ID NO: 5, SEQ ID NO: 6, or SEQ ID NO: 7.

In some embodiments, the inhibitor cocktail comprises one, two, three, four, five, or six inhibitors of CstF64. In some embodiments, the inhibitor cocktail comprises one inhibitor of CstF64. In some embodiments, the inhibitor cocktail comprises three inhibitors of CstF64. In some embodiments, the inhibitor cocktail comprises an siRNA or an shRNA. In some embodiments, the siRNA or shRNA comprises at least 20 nucleotides. In some embodiments, the siRNA or shRNA comprises 20, 21, 22, 23, 24, or 25 nucleotides. In some embodiments, the inhibitor cocktail comprises SEQ ID NO: 1, SEQ ID NO: 2, SEQ ID NO: 3, SEQ ID NO: 4, SEQ ID NO: 5, SEQ ID NO: 6, or any combination thereof.

In some embodiments, the inhibitor cocktail comprises SEQ ID NO: 1, SEQ ID NO: 2, SEQ ID NO: 3, and a pharmaceutically acceptable carrier. In some embodiments, the inhibitor cocktail comprises SEQ ID NO: 1, SEQ ID NO: 2, SEQ ID NO: 4, and a pharmaceutically acceptable carrier. In some embodiments, the inhibitor cocktail comprises SEQ ID NO: 1, SEQ ID NO: 2, SEQ ID NO: 5, and a pharmaceutically acceptable carrier. In some embodiments, the inhibitor cocktail comprises SEQ ID NO: 1, SEQ ID NO: 2, SEQ ID NO: 6, and a pharmaceutically acceptable carrier.

In some embodiments, the inhibitor cocktail comprises SEQ ID NO: 1, SEQ ID NO: 3, SEQ ID NO: 4, and a pharmaceutically acceptable carrier. In some embodiments, the inhibitor cocktail comprises SEQ ID NO: 1, SEQ ID NO: 3, SEQ ID NO: 5, and a pharmaceutically acceptable carrier. In some embodiments, the inhibitor cocktail comprises SEQ ID NO: 1, SEQ ID NO: 3, SEQ ID NO: 6, and a pharmaceutically acceptable carrier. In some embodiments, the inhibitor cocktail comprises SEQ ID NO: 1, SEQ ID NO: 4, SEQ ID NO: 5, and a pharmaceutically acceptable carrier. In some embodiments, the inhibitor cocktail comprises SEQ ID NO: 1, SEQ ID NO: 4, SEQ ID NO: 6, and a pharmaceutically acceptable carrier. In some embodiments, the inhibitor cocktail comprises SEQ ID NO: 1, SEQ ID NO: 5, SEQ ID NO: 6, and a pharmaceutically acceptable carrier.

In some embodiments, the inhibitor cocktail comprises SEQ ID NO: 2, SEQ ID NO: 3, SEQ ID NO: 4, and a pharmaceutically acceptable carrier. In some embodiments, the inhibitor cocktail comprises SEQ ID NO: 2, SEQ ID NO: 3, SEQ ID NO: 5, and a pharmaceutically acceptable carrier. In some embodiments, the inhibitor cocktail comprises SEQ ID NO: 2, SEQ ID NO: 3, SEQ ID NO: 6, and a pharmaceutically acceptable carrier. In some embodiments, the inhibitor cocktail comprises SEQ ID NO: 2, SEQ ID NO: 4, SEQ ID NO: 5, and a pharmaceutically acceptable carrier. In some embodiments, the inhibitor cocktail comprises SEQ ID NO: 2, SEQ ID NO: 4, SEQ ID NO: 6, and a pharmaceutically acceptable carrier. In some embodiments, the inhibitor cocktail comprises SEQ ID NO: 2, SEQ ID NO: 5, SEQ ID NO: 6, and a pharmaceutically acceptable carrier.

In some embodiments, the inhibitor cocktail comprises SEQ ID NO: 3, SEQ ID NO: 4, SEQ ID NO: 5, and a pharmaceutically acceptable carrier. In some embodiments, the inhibitor cocktail comprises SEQ ID NO: 3, SEQ ID NO: 4, SEQ ID NO: 6, and a pharmaceutically acceptable carrier. In some embodiments, the inhibitor cocktail comprises SEQ ID NO: 3, SEQ ID NO: 5, SEQ ID NO: 6, and a pharmaceutically acceptable carrier. In some embodiments, the inhibitor cocktail comprises SEQ ID NO: 4, SEQ ID NO: 5, SEQ ID NO: 6, and a pharmaceutically acceptable carrier.

In some embodiments, the inhibitor cocktail comprises SEQ ID NO: 1, SEQ ID NO: 2, and a pharmaceutically acceptable carrier. In some embodiments, the inhibitor cocktail comprises SEQ ID NO: 1, SEQ ID NO: 3, and a pharmaceutically acceptable carrier. In some embodiments, the inhibitor cocktail comprises SEQ ID NO: 1, SEQ ID NO: 4, and a pharmaceutically acceptable carrier. In some embodiments, the inhibitor cocktail comprises SEQ ID NO: 1, SEQ ID NO: 5, and a pharmaceutically acceptable carrier. In some embodiments, the inhibitor cocktail comprises SEQ ID NO: 1, SEQ ID NO: 6, and a pharmaceutically acceptable carrier.

In some embodiments, the inhibitor cocktail comprises SEQ ID NO: 2, SEQ ID NO: 3, and a pharmaceutically acceptable carrier. In some embodiments, the inhibitor cocktail comprises SEQ ID NO: 2, SEQ ID NO: 4, and a pharmaceutically acceptable carrier. In some embodiments, the inhibitor cocktail comprises SEQ ID NO: 2, SEQ ID NO: 5, and a pharmaceutically acceptable carrier. In some embodiments, the inhibitor cocktail comprises SEQ ID NO: 2, SEQ ID NO: 6, and a pharmaceutically acceptable carrier. In some embodiments, the inhibitor cocktail comprises SEQ ID NO: 3, SEQ ID NO: 4, and a pharmaceutically acceptable carrier. In some embodiments, the inhibitor cocktail comprises SEQ ID NO: 3, SEQ ID NO: 5, and a pharmaceutically acceptable carrier. In some embodiments, the inhibitor cocktail comprises SEQ ID NO: 3, SEQ ID NO: 6, and a pharmaceutically acceptable carrier. In some embodiments, the inhibitor cocktail comprises SEQ ID NO: 4, SEQ ID NO: 5, and a pharmaceutically acceptable carrier. In some embodiments, the inhibitor cocktail comprises SEQ ID NO: 4, SEQ ID NO: 6, and a pharmaceutically acceptable carrier. In some embodiments, the inhibitor cocktail comprises SEQ ID NO: 5, SEQ ID NO: 6, and a pharmaceutically acceptable carrier.

In some embodiments, the inhibitor cocktail comprises SEQ ID NO: 1, and a pharmaceutically acceptable carrier. In some embodiments, the inhibitor cocktail comprises SEQ ID NO: 2, and a pharmaceutically acceptable carrier. In some embodiments, the inhibitor cocktail comprises SEQ ID NO: 3, and a pharmaceutically acceptable carrier. In some embodiments, the inhibitor cocktail comprises SEQ ID NO: 4, and a pharmaceutically acceptable carrier. In some embodiments, the inhibitor cocktail comprises SEQ ID NO: 5, and a pharmaceutically acceptable carrier. In some embodiments, the inhibitor cocktail comprises SEQ ID NO: 6, and a pharmaceutically acceptable carrier. In some embodiments, the inhibitor cocktail comprises SEQ ID NO: 7, and a pharmaceutically acceptable carrier.

In some embodiments, the inhibitor cocktail of any preceding aspect comprises at least 70% sequence identity to SEQ ID NO: 1. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 1.

In some embodiments, the inhibitor cocktail of any preceding aspect comprises at least 70% sequence identity to SEQ ID NO: 2. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 2.

In some embodiments, the inhibitor cocktail of any preceding aspect comprises at least 70% sequence identity to SEQ ID NO: 3. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 3.

In some embodiments, the inhibitor cocktail of any preceding aspect comprises at least 70% sequence identity to SEQ ID NO: 4. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 4.

In some embodiments, the inhibitor cocktail of any preceding aspect comprises at least 70% sequence identity to SEQ ID NO: 5. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 5.

In some embodiments, the inhibitor cocktail of any preceding aspect comprises at least 70% sequence identity to SEQ ID NO: 6. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 6.

In some embodiments, the inhibitor cocktail of any preceding aspect comprises at least 70% sequence identity to SEQ ID NO: 7. In some embodiments, the inhibitor cocktail of any preceding aspect comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to SEQ ID NO: 7.

In some embodiments, the inhibitor cocktail of any preceding aspect further comprises one or more components required to preserve, stabilize, and/or protect the active ingredient of the inhibitor cocktail, wherein the active ingredient comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combination thereof. In some embodiments, the one or more components comprises a buffer (including, but not limited to a saline buffer (such as, for example tris-buffered saline (TBS) or phosphate buffered saline (PBS)), a serum (such as, for example fetal bovine serum (FBS), bovine serum, or donkey serum), an emulsion (such as, for example an oil/water or a water/oil emulsion), an additive, or combinations thereof.

### Methods of Treating, Preventing, Diagnosing, or Detecting Cardiac Fibrosis and Heart Failure

The present disclosure provides methods of treating, preventing, decreasing, reducing, and/or ameliorating a cardiovascular disease including, but not limited to cardiac fibrosis and heart failure, in a subject using an inhibitor of CstF64 or an inhibitor cocktail of CstF64. The present disclosure also provides compositions comprising an inhibitor of CstF64 or an inhibitor cocktail of CstF64 and a pharmaceutically acceptable carrier.

In one aspect, disclosed herein is a method of treating or preventing cardiac fibrosis, comprising administering to a subject in need thereof a therapeutically effective amount of a composition comprising an inhibitor of CstF64.

In one aspect, disclosed herein is a method of treating or preventing heart failure, comprising administering to a subject in need thereof a therapeutically effective amount of a composition comprising an inhibitor of CstF64.

The methods provided herein comprise the composition or inhibitor cocktail of any preceding aspect. In some embodiments, the inhibitor of CstF64 is selected from a nucleic acid or a small molecule. In some embodiments, the inhibitor of CstF64 comprises at least one siRNA. In some embodiments, the inhibitor of CstF64 comprises one, two, or three siRNAs.

In some embodiments, the siRNAs comprises SEQ ID NO: 1, SEQ ID NO: 2, SEQ ID NO: 3, SEQ ID NO: 4, SEQ ID NO: 5, SEQ ID NO: 6, or any combination thereof. In some embodiments, the siRNA comprises SEQ ID NO: 1. In some embodiments, the siRNA comprises SEQ ID NO: 2. In some embodiments, the siRNA comprises SEQ ID NO: 3. In some embodiments, the siRNA comprises SEQ ID NO: 4. In some embodiments, the siRNA comprises SEQ ID NO: 5. In some embodiments, the siRNA comprises SEQ ID NO: 6.

In some embodiments, the inhibitor of CstF64 comprises a shRNA. In some embodiments, the shRNA comprises SEQ ID NO: 7.

The composition of any preceding aspect may be administered in such amounts, time, and route deemed necessary in order to achieve the desired result. The exact amount of the composition of any preceding aspect will vary from subject to subject, depending on the species, age, and general condition of the subject, the severity of the cardiovascular disease, the particular composition of any preceding aspect, its mode of administration, its mode of activity, and the like. The composition of any preceding aspect is preferably formulated in dosage unit form for ease of administration and uniformity of dosage. It will be understood, however, that the total daily usage of the composition of any preceding aspect will be decided by the attending physician within the scope of sound medical judgment. The specific therapeutically effective dose level for any particular subject will depend upon a variety of factors including the cardiovascular disease being treated and the severity of said disease; the activity of the composition of any preceding aspect employed; the specific composition of any preceding aspect employed; the age, body weight, general health, sex and diet of the patient; the time of administration, route of administration, and rate of excretion of the specific composition of any preceding aspect employed; the duration of the treatment; drugs used in combination or coincidental with the specific composition of any preceding aspect employed; and like factors well known in the medical arts.

The composition of any preceding aspect may be administered by any route. In some embodiments, the composition of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition of any preceding aspect (e.g., its stability in the environment of the cardiovascular system), the condition of the subject (e.g., whether the subject is able to tolerate administration), etc.

The exact amount of composition of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

The concentration of active agent(s) can vary widely and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. It will be appreciated that such dosages may be varied to optimize a therapeutic and/or prophylactic regimen in a particular subject or group of subjects.

In one aspect, disclosed herein is a composition of any preceding aspect and a pharmaceutically acceptable carrier selected from an excipient, a diluent, a salt, a buffer, a stabilizer, a lipid, an emulsion, a nanoparticle, and a cream. One or more active agents (e.g. SEQ ID NO: 1, SEQ ID NO: 2, SEQ ID NO: 3, SEQ ID NO: 4, SEQ ID NO: 5, SEQ ID NO: 6, SEQ ID NO: 7, or any combination thereof) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the SEQ ID NO: 1, SEQ ID NO: 2, SEQ ID NO: 3, SEQ ID NO: 4, SEQ ID NO: 5, SEQ ID NO: 6, SEQ ID NO: 7, or any combination thereof can be prepared as a “concentrate”, e.g. in a storage container of a premeasure volume and/or a predetermined amount ready for dilution, or in a soluble capsule ready for addition to a specified volume of water, saline, emulsion, or other diluent.

In some embodiments, the composition of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the composition of any preceding aspect is administered daily. In some embodiments, the composition of any preceding aspect is administered every day, every 2 days, every 3 days, every 4 days, every 5 days, every 6 days, every 7 days, or more. In some embodiments, the composition of any preceding aspect is administered every week, every 2 weeks, every 3 weeks, every 4 weeks, or more. In some embodiments, the composition of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the composition of any preceding aspect is administered every year, every 2 years, every 3 years, every 4 years, every 5 years, or more.

In some embodiments, the cardiovascular disease includes, but is not limited to cardiac arrest/heart failure, congestive heart failure, hypertensive heart disease, diabetic hypertrophic cardiomyopathy, idiopathic dilated cardiomyopathy, aortic stenosis, coronary artery disease, high/low blood pressure, congenital heart defects/diseases (including, but not limited to atrial septal defects, atrioventricular septal defects, coarctation of the aorta, double-outlet right ventricle, d-transposition of the great arteries, Ebstein anomaly, hypoplastic left heart syndrome, and interrupted aortic arch), arrhythmia, peripheral artery disease, stroke, cerebrovascular disease, renal artery stenosis, aortic aneurysm, cardiomyopathies, pulmonary heart disease, cardiac dysrhythmias, endocarditis, inflammatory cardiomegaly, myocarditis, eosinophilic myocarditis, valvular heart diseases, rheumatic heart diseases, and other related cardiovascular diseases.

In one aspect, disclosed herein is a method of diagnosing a subject with heart failure or cardiac fibrosis, comprising obtaining a biological sample from a subject, determining an expression level of CstF64, and determining the subject has heart failure if the expression level of CstF64 is increased in the subject in comparison to a control sample.

In one aspect, disclosed herein is a method of treating a subject with heart failure or cardiac fibrosis, comprising obtaining a biological sample from a subject, determining an expression level of CstF64, determining the subject has heart failure if the expression level of CstF64 is increased in the subject in comparison to a control sample, and administering a therapeutic agent. In some embodiments, the therapeutic agent is an inhibitor of CstF64. In some embodiments, the therapeutic agent is an siRNA inhibitor of CstF64. In some embodiments, the therapeutic agent comprises SEQ ID NO: 1, SEQ ID NO: 2, SEQ ID NO: 3, SEQ ID NO: 4, SEQ ID NO: 5, SEQ ID NO: 6, or any combination thereof.

In one aspect, disclosed herein is a method of treating a subject with heart failure or cardiac fibrosis, comprising obtaining a biological sample from a subject, determining an expression level of CstF64, determining the subject has heart failure if the expression level of CstF64 is increased in the subject in comparison to a control sample, and administering an inhibitor of CstF64.

In some embodiments, the biological samples includes, but is not limited to a blood sample, a serum sample, a plasma sample, a cerebral spinal fluid (CSF) sample, or a saliva sample.

In some embodiments, expression level of CstF64 is increased by 10%, 15%, 20%, 25%, 30%, 35%, 40%, 45%, 50%, 55%, 60%, 65%, 70%, 75%, 80%, 85%, 90%, 95%, 99%, 100%, or any percentage in-between the given range relative to a control sample.

In some embodiments, the expression level of CstF64 is measured by mRNA levels. In some embodiments, CstF64 mRNA levels are measured using one or more methods including, but not limited to polymerase chain reaction, or any derivative thereof, RNA sequencing, northern blot assays, nuclease protection assays (NPA), next generation sequencing (NGS), and in situ hybridization. In some embodiments, the expression level of CstF64 is measured by protein levels. In some embodiments, CstF64 protein levels are measured using one or more methods including, but not limited to western blot assays, mass spectrometry, direct enzyme-linked immunosorbent assay (ELISA), indirect ELISA, high-performance liquid chromatography (HPLC), liquid chromatography-mass spectrometry, immunoprecipitation, and immunostaining.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Impact of 3′UTR Alternative Polyadenylation (APA) in Cardiac Fibrotic mRNAs

Heart failure (HF) is a devastating disease with a poor prognosis and few therapeutic options and an unmet medical need. It is estimated that in 2030, more than 8 million people in the United States will have HF and that the associated financial burden for direct medical costs will exceed $50 billion. In the setting of non-ischemic cardiomyopathies, nearly half of the cases of HF are associated with diffuse interstitial fibrosis. In this regard, cardiac fibrosis is a major driver associated with the growing burden of HF. Cardiac fibrosis, is characterized by excess deposition of fibrous extra cellular matrix protein (ECM) proteins, which produces myocardial stiffness, cardiac remodeling, and eventual diastolic and systolic dysfunction and eventually HF. Cardiac fibroblasts are the primary cells involved in fibrotic responses. Following cardiac injury, cardiac fibroblasts differentiate into highly specialized and contractile myofibroblasts, expressing alpha-smooth muscle actin (SMA) which produce fibrillar collagen and secrete matricellular proteins. Cardiac fibrosis disease severity can be reduced by targeting myofibroblasts in experimental models. However, the mechanisms underlying cardiac fibrosis and HF are not well understood, which may be explained by its high tissue heterogeneity and clinical diversity. Thus, identification of the molecular mechanisms inducing and maintaining myofibroblast activation is to guide strategies for the design of effective therapies for HF and other fibrotic diseases.

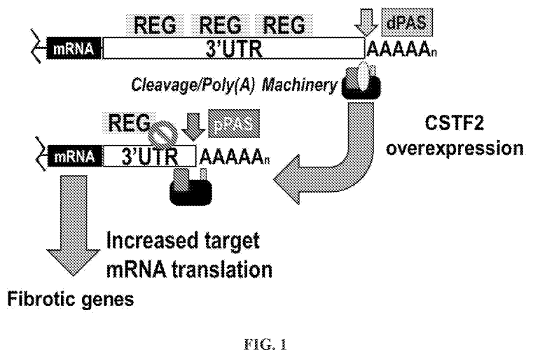

Polyadenylation is crucial to the stabilization of mature mRNA in a way that potentiates translation. Recent discoveries using transcriptome-wide techniques have revealed that most human genes contain more than one poly(A) site, thereby indicating that alternative polyadenylation (APA) is a widespread phenomenon that generates transcript isoforms with varying lengths of 3′ untranslated regions (3′UTRs). Through alteration of 3′UTRs, APA regulates the stability, cellular localization, and translation efficiency of target RNAs, because 3′UTRs are docking platforms for factors that control these regulatory layers (e.g., microRNAs (miRNAs) and RNA-binding proteins). The distal canonical polyadenylation site (dPAS, FIG. 1) that is located at the distal side of the 3′UTR is generally the most efficient and is more frequently used in normal cells; however, in highly proliferating or differentiating cells, there is a global distal-to-proximal shift in APA site selection (pPAS, FIG. 1), generating transcripts with shorter 3′UTRs, which evade microRNA recognition, thus more stable and having increased protein translation.

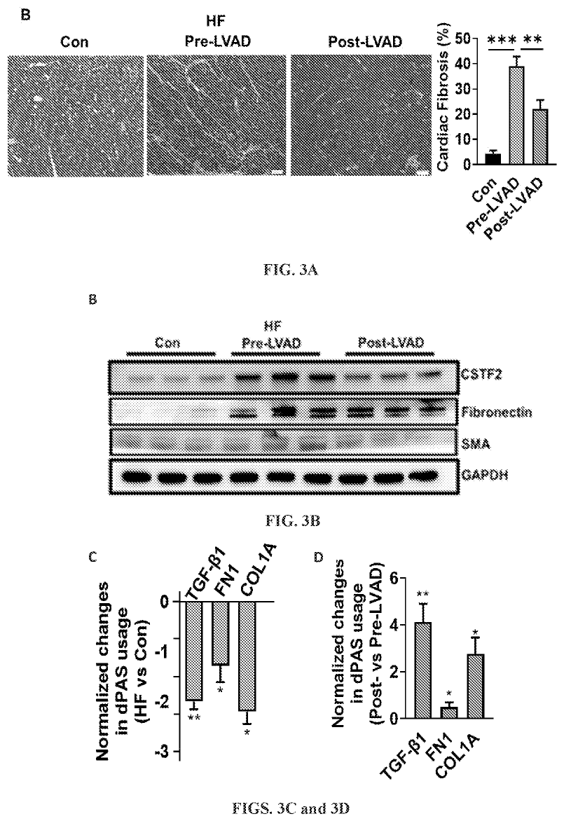

The selection of poly(A) sites can be determined not only by adjacent sequence elements, but also the levels of polyadenylation protein complexes. Several protein complexes are needed for poly(A) signal recognition, cleavage, and addition of poly(A) tails, including CstF (Cleavage stimulation factor), CFIm (cleavage factor Im) and CFIIm (cleavage factor IIm). Importantly, up regulation of CSTF2, also known as CSTF64, a key component of CSTF complex recognizing U/GU motifs downstream of the cleavage site, induces global shortening of messenger RNAs and is recognized as a novel mechanism that increases the expression of several known oncogenes and enhances cancer cell proliferation and differentiation. Findings demonstrate that CSTF2, the RNA binding component of the CstF complex is up-regulated in myofibroblasts found in fibrotic hearts and show that its up-regulation is directly associated with increased APA and increased expression of pro-fibrotic factors (FIG. 3-6). This example demonstrates alternative polyadenylation (APA) in cardiac fibrosis. This example further defines the role of CSTF2 in the pathogenesis of cardiac fibrosis and utilizes RNA-Sequencing (RNA-Seq) as a tool to systemically identify pro-fibrotic factors that are directly targeted by CSTF2 and use APA as a mechanism to enhance their expression. This example also demonstrates the amplification of pro-fibrotic factors in heart failure (HF). Given the general lack of available therapies for HF, the present disclosure provides methods to dampen the over-production of pro-fibrotic factors in HF.

The present example provides the following concepts and methods:
1. That CSTF2 up-regulation in cardiac fibroblast shortens 3′UTRs of the ECM genes, and thereby amplifies cardiac fibrosis.
2. The identification of a 3′UTR shortening pathway in cardiac fibrosis. Currently, little is known about the role of alternative polyadenylation (APA) and 3′UTR shortening in human cardiac disease.
3. Integration of animal models and human samples in mechanistic studies. Using plasma, freshly explanted heart tissue and cardiac fibroblast from patients with a diagnosis of cardiac fibrosis. These resources are used to examine CSTF2 expression and APA in cardiac fibrosis and correlate with findings from the mouse model.
4. Mechanisms leading to CSTF2 up-regulation in cardiac fibrosis: Data shows that transforming growth factor beta 1 (TGFβ1) treatment leads to upregulation of CSTF2 levels in cardiac fibroblast. This process is modulated by the inhibition of miR-124 that are capable to up-regulate CSTF2 expression in culture.
5. Techniques monitoring gene expression and APA in cardiac fibrosis: Poly(A)-ClickSeq (PAC-seq), together with new algorithms to globally identify APA in mRNAs is a state-of-the-art-method that provides a large-scale view of the mechanism(s) by which how CSTF2 up-regulation in cardiac fibroblast leads to cellular and molecular changes.
6. Using conditional CSTF2 knockdown mice as a tool to understand the role of APA and 3′UTR length in cardiac fibrosis. The pathological consequences of 3′UTR shortening in cardiac fibrosis is addressed. Conditional knockdown mice with depleted CSTF2 in cardiac fibroblast were examined for effects on 3′ UTR shortening.
7. The identification of genes that undergo APA following CSTF2 up-regulation in cardiac fibroblast. PAC-seq approaches uncover pathways that are altered following CSTF2 up-regulation in cardiac fibroblasts. Profibrotic genes with shorten 3′UTR length are identified to be altered following CSTF2 up-regulation.
8. The animal models, surgeries, functional analysis, histology, molecular analysis, RNA-seq analysis, and statistical procedures that are used have all been validated and verified before. All analyses are carried out in a blinded fashion. Sex as a biological variable, both sexes are included. Unbiased results are achieved by randomly assigning each animal to a protocol. Blinded echocardiography and histology scoring are performed. Control and experimental samples are included on the same blots and to stain the immunohistochemistry samples together. Image acquisition is conducted using the same gain and exposure factors. Statistical analyses are performed using GraphPad Prism, including D'Agostino-Pearson normality testing. A paired t-test or an ANOVA with a Bonferroni post hoc test is performed to compare groups between them. A P value of 0.05 is considered significant. A power analysis is used to calculate the minimum number of experiments that is needed to achieve a power of (1−β) of 0.85 with a Type I error (α) probability of <0.05.

### Results

### Alternative Polyadenylation (APA) is Differentially Regulated in Heart Failure (HF) Compared to the Healthy Heart.

Human tissue samples were obtained from failing human hearts at the time of cardiac transplantation and paired samples of myocardial tissue were collected from patients during left ventricular assisted device (LVAD) insertion (pre-LVAD) and at the time of LVAD removal during heart transplantation at the Houston Methodist DeBakey Heart & Vascular Center, Houston Methodist Hospital. The tissues were immediately frozen in liquid nitrogen and stored at −80 °C until use. Normal tissue samples were obtained from donor hearts that were not used for transplantation and were collected and stored in the same manner.

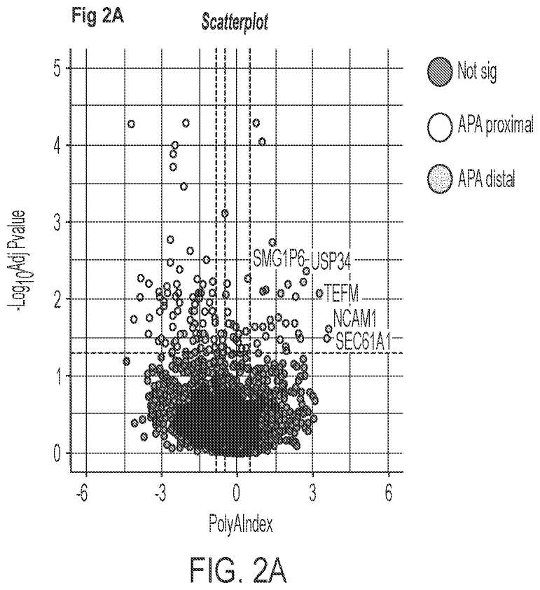

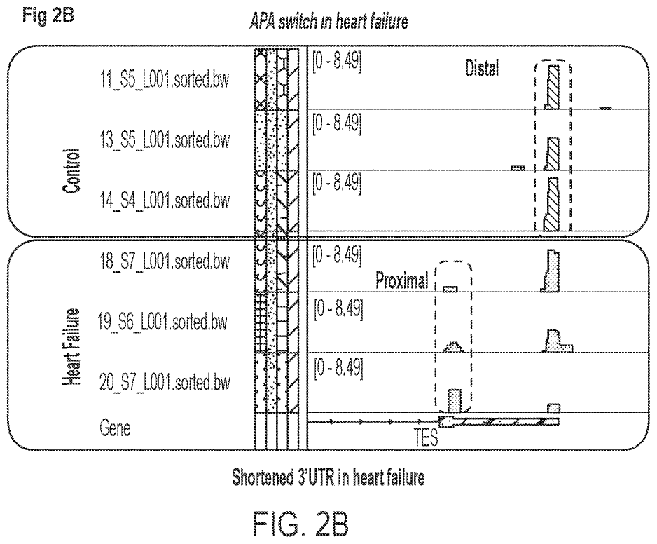

To understand whether a global alternative polyadenylation (APA) event is presented in the progression of heart failure (HF), the APA profiles of failed human heart were characterized and compared to healthy hearts by using PAC-seq the PolyA-miner. Shortening versus lengthening of 3′UTRs was determined based on the PolyA index, a metric unit that determines the length of 3′UTR. A positive PolyAIndex value shows the lengthening of 3′UTR while a negative value shows the shortening of 3′UTR. The APA analysis revealed 96 genes with shortened 3′ UTR and 33 genes with lengthened 3′ UTR (FIG. 2A). Many of these genes have been reported to play a role in the HF before. This shows that the polyadenylation events in these hearts are disease specific. In the UCSC (University of California Santa Cruz) genome browser, APA profiles can be directly visualized as the number of reads. The peaks that represent the total number of reads supporting cleavage at each position along the transcript can be visualized in the genome browser. A shortened gene is shown as an example in FIG. 2B. FIG. 2B reveals one distal cleavage site in the three healthy hearts (FIG. 2B) while the three HF specimens show the usage of a proximal cleavage site as well (FIG. 2B, lower panel). Such usage of proximal cleavage site shortens the 3′ UTR length and escapes the regulation by miRNAs and RNA binding proteins. APA machinery genes show decreased expression in the left ventricle of HF patients. These findings show that the 3′-UTR length of numerous mRNAs is altered in the failing human heart.

### APA Analysis Identifies Fibrotic Pathways Targeted by 3′ UTR Shortened Genes in the HF Patients.

The 3′ UTR shortened genes causes them to lose regulation by miRNA or RNA binding proteins and ultimately promotes enhanced protein expression for them. The pathway enrichment of these 96 shortened genes revealed that these genes were involved in heart disease related pathways including fibrosis of myocardium. Since one of the top enriched pathways of these shortened genes was related to the fibrosis, it showed that the APA events in HF cause shortening of profibrotic genes, enhance their expression, and progress fibrosis. This observation is further corroborated by the Masson's trichrome staining of the cross-section of heart tissue from HF patients. The staining showed significantly increased fibrosis compared to the cross-section of tissue from the healthy patients (FIG. 3A). These findings show that the APA events enriched fibrotic pathways in the failing human heart.

Correlation of APA with Recovery of HF Patients on Left Ventricular Assisted Device (LVAD) Support.

It was shown that the beneficial effects of left ventricular assisted device (LVAD) support is in part due to improved myocardial energetics, and/or myocyte survival. Following the observation that enriched fibrosis related pathway from shortened genes and increased fibrosis in the HF, it was thought that the 3′UTR shortening of profibrotic genes is associated to trans-acting protein complexes involved in cleavage and polyadenylation. To test this, the expression of CSTF2 was investigated in healthy, pre-LVAD (HF), and post-LVAD (recovered) hearts. The left ventricular tissue from a cohort of 5 patients' pre and post LVAD support was assessed for a mean of 295 days and quantified interstitial fibrosis by Masson Trichrome stain. CSTF2 protein expression was significantly increased (1.8±0.3116 densitometry CstF64/GAPDH, p<0.01) in pre-LVAD HF tissues compared to healthy tissues showed. After LVAD support, interstitial fibrosis was significantly reduced compared to pre-LVAD support (FIG. 3A). The reduction in interstitial fibrosis was associated with a significant decrease in CSTF2 expression and decrease of fibronectin 1 (FN1) and SMA (p<0.05) (FIG. 3B). These histological changes were associated with an improved cardiac function. To find if fibrosis genes were regulated by APA during the progression of HF and recovery post-LVAD insertion, RT-qPCR was used to calculate the percentage of mRNA that are using the distal Poly-A site (dPAS). Two sets of primers were designed, one targeting the open reading frame which quantifies the total transcripts and other targeting near the distal PAS which quantifies the transcripts with longer 3′UTR. Using this method, the major fibrosis associated genes were shown to be shortened in HF compared to controls and lengthened in post-LVAD recovery compared to pre-LVAD HF (FIGS. 3C and D). These findings show that fibrosis correlates with 3′-UTR shortened ECM genes in the failing human heart, which is paralleled by an increased expression of CSTF2.

### APA Regulates Fibrosis in the Non-Ischemic Mouse Model of HF.

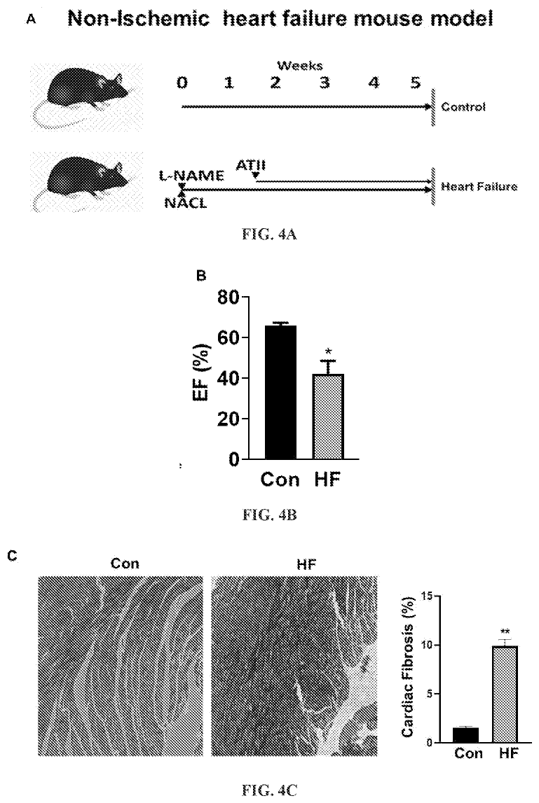

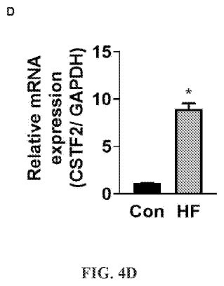

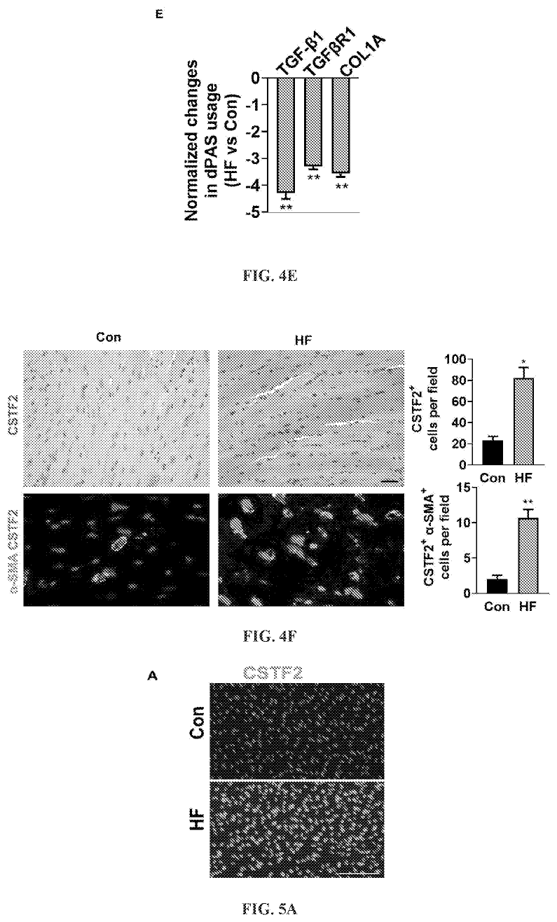

The mouse model where C57/BL6 mice underwent HF induction over a 5-week period was used (FIG. 4A). At the end of this 5-week period (HF induction phase), mice develop a phenotype of HF including cardiac fibrosis and a reduction of ejection fraction (FIG. 4B). The Masson's trichrome staining of the cross-section of heart tissue from HF mice showed significantly increased fibrosis compared to control mice (FIG. 4C). Then, the expression of CSTF2 and fibrogenic genes was examined in the heart tissue from the control and HF mice. The HF mice hearts showed increased expression of CSTF2 by qPCR like that of human HF (FIG. 4D). The dPAS changes of major fibrosis genes such as TGFβ1, TGFβR1, and COL1A which were shortened in HF mice hearts correlating with the increased expression of CSTF2 (FIG. 4E). Finally, it was confirmed that the increased expression of CSTF2 at protein level in HF mice hearts by immunohistochemistry (FIG. 4F). The dual immunofluorescence staining of CSTF2 and α-SMA showed significantly increased expression of CSTF2 in α-SMA-positive myofibroblasts in HF. These results showed that the differential APA regulation is targeting the profibrotic genes in the mouse model of HF as well.

Isolated Cardiac Fibroblasts from HF Patients Show Increased Expression of CSTF2 and 3′UTR Shortening of Key Profibrotic Genes.

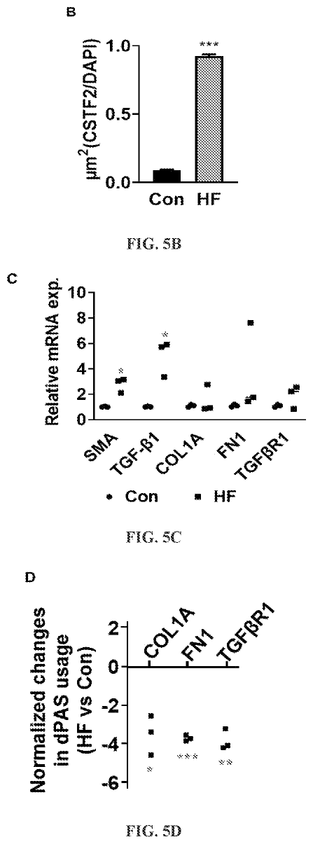

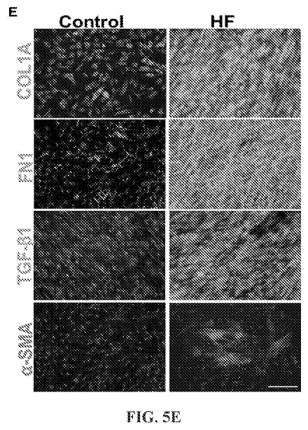

The cardiac fibroblasts were isolated from the human HF patients to validate previous findings. The immunofluorescence staining showed a significantly higher expression of CSTF2 in cardiac fibroblasts from HF patients than the controls (FIG. 5A, B). The transcript levels of α-SMA and TGFβ1 were significantly higher in cardiac fibroblasts from HF patients. But not all the HF cardiac fibroblasts showed significant differences in COL1A, FN1, and TGFβR1 expression compared to the healthy controls (FIG. 5C). On the other hand, the investigation of dPAS usage showed that there is a significant 3′UTR shortening in COL1A, FN1, TGFβR1 in cardiac fibroblasts from HF compared to healthy controls (FIG. 5D). The immunofluorescence staining of HF fibroblasts showed significantly increased expression of COL1A, FN1, TGFβ1, and α-SMA compared to the healthy controls (FIG. 5E). These data showed that irrespective of the total transcript levels in HF and control fibroblasts, 3′UTR shortening is sufficient to enhance the protein expression of those fibrotic genes.

### CSTF2 Knockdown Lengthens the 3′UTR of Profibrotic Genes in TGFβ1 Treated Human Cardiac Fibroblasts.

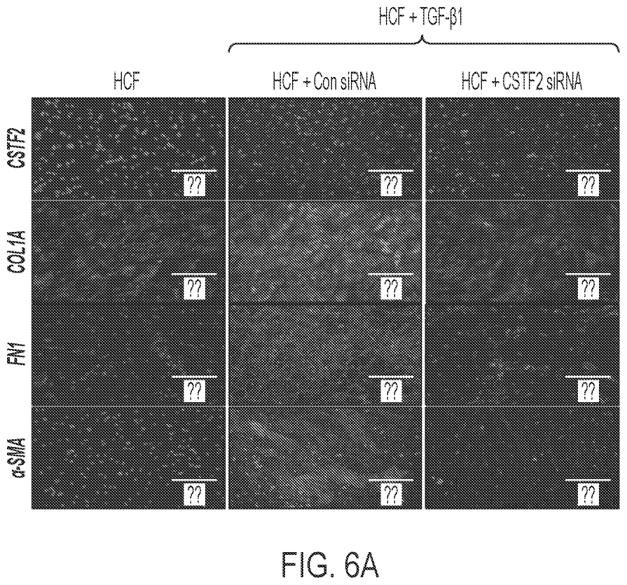

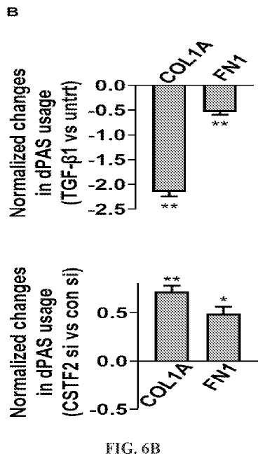

The APA events and CSTF2 dysregulation was investigated in a well-established model of in vitro fibrosis. The human control cardiac fibroblasts were treated with 10 ng/ml of TGFβ1 for 72 hours. The TGFβ1 treated human cardiac fibroblasts showed excessive deposition of COL1A (2.489±0.1052 μm² norm to DAPI, p<0.01) and FN1 (4.046±0.1068 μm² norm to DAPI, p<0.001) as well as significantly greater number of myofibroblasts marked by α-SMA (8.653±1.024 μm² norm to DAPI, p<0.05) compared to the controls. So, it was investigated whether this myofibroblast transition is a result of 3′UTR shortening in the treated fibroblasts. The immunofluorescence staining of TGFβ1 treated fibroblasts showed significant up-regulation of CSTF2 (0.9018±0.01912 μm² norm to DAPI, p<0.001) compared to the untreated fibroblasts. So, the expression of CSTF2 was efficiently knocked down (0.7153±0.0205 μm² norm to DAPI, p<0.001) from the TGFβ1 treated fibroblasts and showed significant reduction in deposition of COL1A (2.112±0.1632 μm² norm to DAPI, p<0.001), FN1 (3.315±0.1514 μm² norm to DAPI, p<0.001), and α-SMA (7.20=1.038 μm² norm to DAPI, p<0.05) compared to treated fibroblasts transfected with scrambled siRNA (FIG. 6A). In addition, the dPAS usage in these treated fibroblasts showed significant shortening in COL1A and FN1 which correlated with its increased protein expression and lengthening in CSTF2 knockdown fibroblasts which correlated with its reduced protein expression after the knockdown in TGF-β1 treated fibroblasts (FIG. 6B). Hence, changes in 3′UTR length in the COL1A and FN1 genes correlated with their protein expression.

### Overexpression of CSTF2 Leads to 3′UTR Shortening of Fibrotic Genes in Mouse Cardiac Fibroblasts.

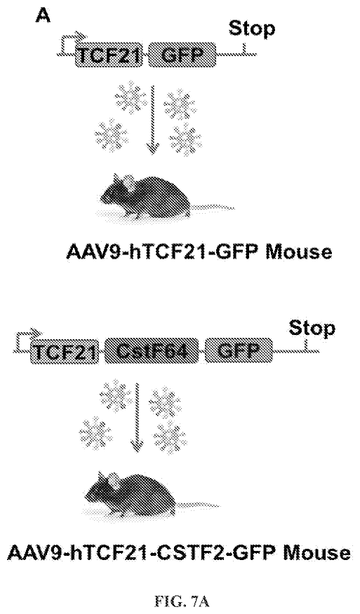

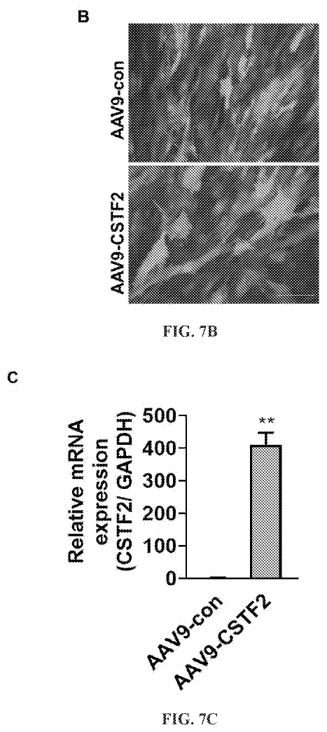

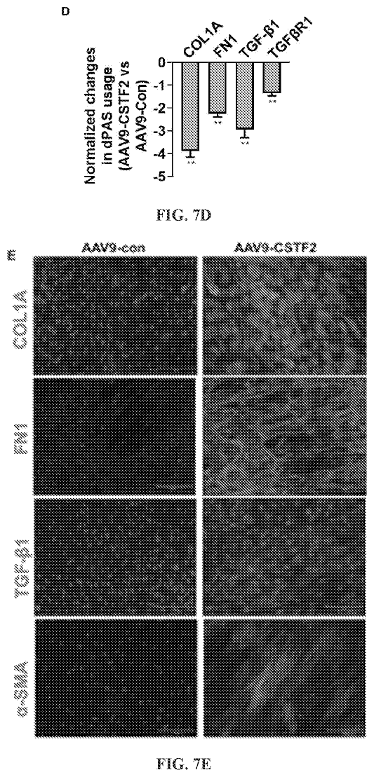

The expression data from the mouse model of HF strongly showed the shortening of profibrotic genes as a consequence of the upregulation of CSTF2. The overexpression of CSTF2 is related to the global APA changes in myofibroblasts thus effecting the genes involved in the pathogenesis of cardiac fibrosis. To investigate if CSTF2 directly plays a role in myofibroblast transition to promote fibrosis, CSTF2 was overexpressed in control human cardiac fibroblasts with the help of an adeno-associated virus 9 (AAV9) consisting of CSTF2 expression construct under human TCF21 promoter (AAV9-CSTF2) VectorBuilder (Chicago, IL). The fibroblasts were successfully transfected with AAV9-control and AAV9-CSTF2 (FIG. 7A, B). The transcript levels of CSTF2 after the infection with AAV9-CSTF2 was more than 300-fold compared to that of controls (FIG. 7C). With CSTF2 overexpressed, the investigation of dPAS usage showed that there is a significant 3′UTR shortening in COL1A, FN1, TGF-β1, and TGFβR1 in AAV9-CSTF2 fibroblasts compared to AAV9-control fibroblasts (FIG. 7D). Finally, the immunofluorescence staining of AAV9-CSTF2 fibroblasts showed significantly increased expression of COL1A, FN1, TGFβ1, and α-SMA compared to the AAV9-control (FIG. 7E). These data showed that overexpression of CSTF2 is sufficient to induce 3′UTR shortening in fibrosis genes to enhance the protein expression and induce myofibroblast transition.

Taken together, these results show that CSTF2 is up-regulated in myofibroblasts in human and fibrotic mouse hearts (FIGS. 3C and 4D, and FIG. 4), and that simple in vitro CSTF2 overexpression increases the expression of fibrotic factors (FIGS. 6 and 7), showing that the detrimental effects of CSTF2 up-regulation are mechanistically linked to the pathogenesis of cardiac fibrosis-enhancing the production of fibrotic factors through APA.

### Characterizing CSTF2 in Human Cardiac Fibroblasts and Identifying Fibrotic Genes Deregulated by APA.

An important aspect herein is the evaluation of CSTF2 expression levels in patients with a diagnosis of cardiac fibrosis. This is essential to the correlation of the expression of CSTF2 with failure and recovery. Through the Houston Methodist Hospital Heart Bio-bank there is access to approximately 500 heart explants from patients with chronic heart diseases, including paired samples of myocardial tissue from patients during LVAD insertion (pre-LVAD) and LVAD removal during heart transplantation (post-LVAD). The availability of this bio-bank provides an immense resource to study the cardiac fibrosis and recovery of HF in disease-relevant tissues.

First, the CSTF2 protein levels are determined (by western blot) in normal and failing hearts. For failing hearts, tissue from patients with confirmed cardiac fibrosis are used. A striking reduction in interstitial myocardial fibrosis was noted, with a concomitant decrease in CSTF2 after LVAD explanation (FIG. 3C). Hence, changes in APA events explain, in part, the improvement in HF patients on LVAD support. Immunoblots from the ventricles of multiple pre and post-LVAD samples are used to assess fibrosis and CSTF2 expression. RT-qPCR is performed to evaluate dPAS usage to confirm the 3′UTR shortening or lengthening of target genes. Finally, RNA-seq is performed using PAC sequencing technology that allows identification of 3′UTR changes through direct 3′ end sequencing. These data are analyzed using DaPars and PolyA-miner algorithm to reveal global 3′UTR changes and subsequently the pathways involved.

Temporal expression of CSTF2 in experimental HF and recovery model. A mouse model of non-ischemic HF and recovery is used to assess temporal expression of CSTF2. It is known that non-ischemic HF patients comprise the other 50% of HF cases. In these cases, a disseminated interstitial fibrosis predominates, with reduced ventricular compliance and function; this model emulates this form of HF. After 5 weeks, the HF recovery is associated with reduced myocardial fibrosis, and improved ventricular function that mimics the observations in pre and post-LVAD patients. Most HF patients who manifest reversal of HF after LVAD have a non-ischemic etiology. CSTF2 expression is increased in the failing mouse ventricles (FIG. 3B). To document the induction of HF and recovery, ventricular function is non-invasively monitored by echocardiography in sham and experimental mice from 0-9 weeks. On weeks 0-5 of the HF phase and weeks 6-9 of the HF recovery phase, the heart is excised to obtain myocardial tissue. Interstitial fibrosis is assessed by trichrome staining and the extent of ECM remodeling by immunohistochemistry for fibrosis markers mentioned in Table 1. Further, lysate from ventricles are used to visualize the expression levels of CSTF2 by immunoblot. Various genes expression which are altered in fibrosis are tested by qRT-PCR (see Table 1) to confirm dPAS usage. Finally, PAC-Seq is performed on hearts excised at weeks 0-9 to reveal the global APA changes.

PAC-seq and 3′UTR analysis from isolated cardiac fibroblasts. Fibroblasts from human heart tissues are isolated similarly to mice as previously described and RNA-Seq is performed to reveal genes with 3′UTR changes. This example demonstrates that upregulation of CSTF2 leads to 3′UTR shortening and to identify pathways involved. Furthermore, it also validated changes in the expression of ECM mediators that were altered by CSTF2 overexpression. This is performed using dPAS usage calculation, determining gene expression levels as described in FIG. 3D, and with conventional immunoblots. Importantly, in using human cardiac fibroblasts, the clinical data available is used to include left-heart catheterization data and cardiac function tests to help link 3′UTR with clinical status. This provides correlated pre-clinical findings with specimens of human heart failure.

Identifying the Mechanism Underlying CSTF2 Reduction and Determining their Use as a Therapeutic Target for Cardiac Fibrosis.

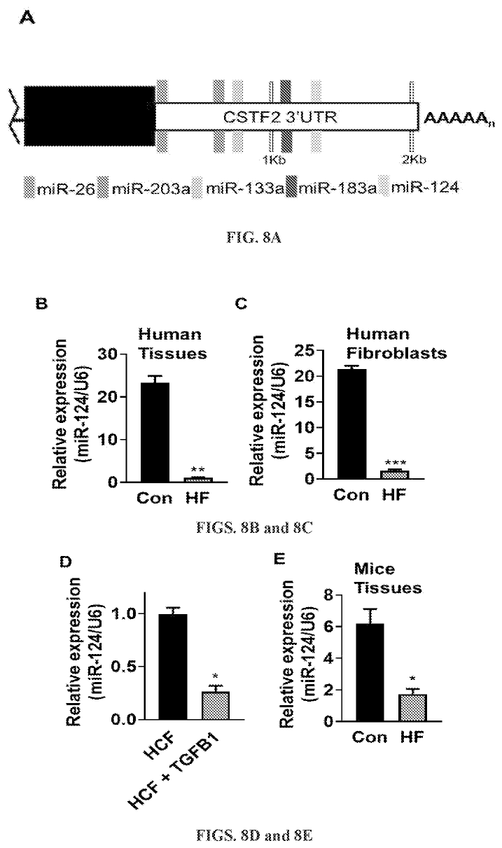

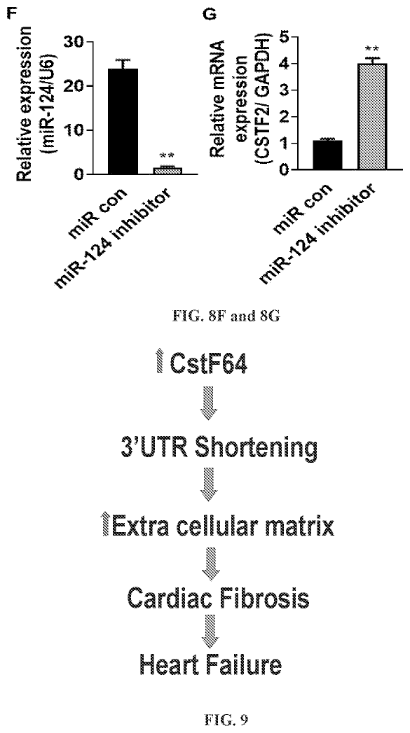

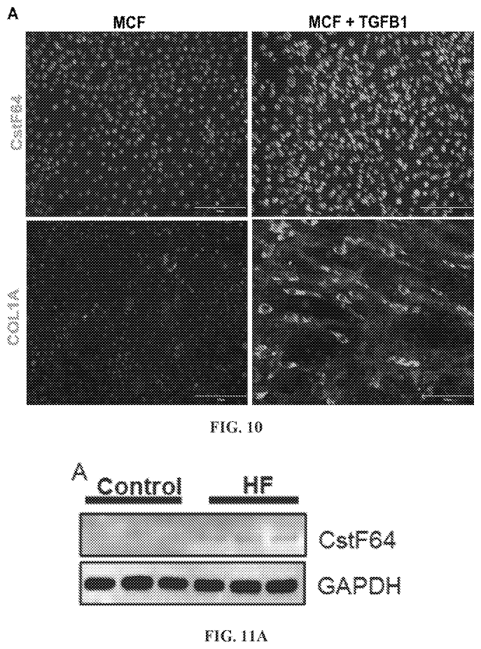

The mechanisms directing CSTF2 upregulation in cardiac fibroblasts are unknown. Understanding these mechanisms, strategies are being developed for preventing or eliminating the upregulation of CSTF2 during active fibrosis. To identify the factors that regulate CSTF2, miRs that target CSTF2 were screened. According to TargetScan, five different microRNAs (miR-124, miR-203a, miR-183a, miR-133a, and miR-26) have at least 2 binding sites within the 3′UTR of CSTF2 (FIG. 8A). It is relevant that miR-124 targeting CSTF2 was found to be decreased in human fibrotic hearts (FIG. 8B) and isolated cardiac fibroblasts (FIG. 8C). Further, qRT-PCR is used to measure the levels of this microRNA in control human cardiac fibroblasts with and without TGFβ1 treatment and found that miR-124 was down-regulated following TGFβ1 treatment and overexpressing the CSTF2 levels (FIG. 8D). Finally, the mice HF tissues also showed downregulation of miR-124 by at least 3-fold (FIG. 8E). Next, to directly test if this miRNA impacts the up-regulation of CSTF2, miR-124 was inhibited by transfecting cardiac fibroblasts with miR-124 inhibitors followed by measuring CSTF2 levels and found that inhibition of miR-124 up-regulated CSTF2 (FIG. 8F,G). These observations clearly point at TGFβ1-induced miR regulation of CSTF2 expression.

Evaluation of miR-124 expression in fibrotic hearts. TGFβ1 treatment results in down regulation of miR-124, which increases CSTF2 expression. The TGFβ1 is a critical factor that contributing to cardiac fibrosis. Interestingly, findings in FIG. 8 show that miR-124 is decreased in human and mice fibrotic hearts. As such this data is strengthened by first examining the levels of miR-124 in patient fibrotic samples, and in the multiple normal and HF fibroblast cell lines. In addition, to examine the conservation of this response in mice, measure miR-124 levels are measured in primary fibroblasts derived from control and HF mice. In addition, the expression of miR-124 is examined at different times during the development of cardiac fibrosis in the HF mouse model. Next, in situ hybridization is used to examine the localization of miR-124 in the hearts of normal and HF patients and in control and HF mice. In addition, temporal expression of miR-124 is checked in cardiac fibroblast isolated from pre- and post-LVAD patients. This information is useful for correlating miR-124 with CSTF2 levels and provides important insight for understanding the upregulation miR-124 expression in vivo.

Testing miR-124 mimics to inhibit CSTF2 up-regulation in vitro. To examine whether CSTF2 3′-UTR is indeed a target of this miRNA, a Dual-Luciferase 3′-UTR reporter assay is performed in vitro. HEK293T cells are transfected with miR-124 and with the dual-luciferase vectors to monitor the effect of miRNAs on firefly luciferase expression. Similar experiments are repeated in human cardiac fibroblasts to confirm miR-124 mediated regulation of CSTF2. These experiments provide in vitro evidence to help determine whether miR-124 binds to and regulates CSTF2 in human cardiac fibroblast. This data shows that miR-124 is decreased following TGFβ1 treatment which increases CSTF2 (FIG. 8D). The capacity of mimics directed to increase miR-124 to prevent the increase of CSTF2 in cardiac fibroblast treated with TGFβ1 is tested. Nanoparticles labeled with the fluorescent probe Cy5.5 was developed and successfully delivered to failing hearts in non-ischemic HF mouse model and cardiac fibroblasts. Nanoparticle localization in the HF hearts occurred with the greatest signal in areas of fibrosis. Nanoparticle uptake was shown in the cytoplasmic region of cultured fibroblasts with DAPI nuclear stain. So, nanoparticles can be delivered to fibrotic regions of the heart in HF mice, demonstrating enhanced drug delivery to specific cells in this diseased region of the heart and decreased systemic effects. Here, the nanoparticles are loaded with miR-124 mimic to increase miR-124 directed regulation in the 3′UTR of CSTF2 and decrease CSTF2 levels. The capacity of these nanoparticles loaded with miR-124 mimic are evaluated for depletion of protein expression of CSTF2 (measured by immunoblots) from cardiac fibroblast exposed to TGFβ1. This is also performed in HF cardiac fibroblasts that have heightened activation of these pathways. This helps to clarify how TGFβ1 regulates CSTF2 up-regulation and APA in cardiac fibroblasts. These mimics have successfully been used in approaches in different cardiac cells with data showing positive results in the overexpression of miR124 in cardiac fibroblast. Importantly, PAC-seq and subsequent daPARS and Poly Aminer analyses are performed to determine whether depletion of CSTF2 expression reverses 3′UTR-shortening of affected transcripts; various genes expression (see Table 1) is measured by qRT-PCR to confirm dPAS usage.

An in vivo evaluation of the effect of miR mimics on CSTF2-dependent APA in cardiac fibrosis. The association of CSTF2 up-regulation with increased fibrotic mediator production in cardiac fibrosis is associated shows that a clinical benefit is associated with reversing its upregulation. Towards this end, the effects of overexpression miR-124 levels with mimics is examined during various stages of established fibrosis in the HF mouse model.

To evaluate the capacity of overexpressing miR-124 for the treatment of cardiac fibrosis, treatment with the Cy5.5 nanoparticle (control) or miR-124 mimic encapsulated Cy5.5 nanoparticle is initiated once weekly for 4 weeks by intraperitoneal injection to prevent the development of cardiac fibrosis. The functional and molecular endpoints used to determine pathology and fibrotic remodeling are performed as previously described. Next, the therapeutic capacity of miR mimics to treat established cardiac fibrosis and remodeling are evaluated. Here, mice with or without HF are treated with control or mi-R124 mimics beginning on day 28 as described above. Mice are sacrificed on day 28 and functional and molecular endpoints used to determine pathology and fibrotic remodeling are performed as previously described. Immunohistochemistry for α-SMA and CSTF2 is used to assess the specificity of CSTF2 inhibition in myofibroblasts during fibrosis. Importantly, CSTF2 immunoblots from hearts and isolated cardiac fibroblasts, and various genes expression which is altered in fibrosis are tested by qRT-PCR (see Table 1) to confirm dPAS usage. Together, these data provide additional evidence supporting a role for miR-124 in the regulation of CSTF2 as well as provide insight into ways to manipulate this pathway in the context of cardiac fibrosis.

### Determining the In Vivo Role of CSTF2 in Cardiac Fibrosis.

Results here show that increased CSTF2 expression in failing human and mice fibrotic heart tissue as well as in myofibroblasts (FIG. 4). Additionally, CSTF2 depletion abrogated TGFβ_induced ECM and fibrotic genes by modifying its 3′UTR length. It is thought that fibroblast specific CSTF2 inhibition lengthen 3′UTR of ECM genes and inhibit cardiac fibrosis by decreasing the production of fibrotic factors (FIG. 1). Taken together, these results show that CSTF2 upregulation worsens cardiac fibrosis and CSTF2 inhibition using short hairpin RNA (shRNA) reduces cardiac fibrosis and improves heart function. Here, the extent to which alterations in CSTF2 can alter 3′UTR length and enhance resolution of ECM matrix remodeling and inhibit cardiac fibrosis is tested with fibroblast CSTF2 as the focus.

Using AAV9-hTCF21-CSTF2 ShRNA-GFP mice and Ang II/LNAME/NaCl. Here, alterations of CSTF2 is evaluated to determine whether influence on 3′UTR length of ECM mediated cardiac fibrosis in HF mice. Since 3′UTR shortening of ECM genes was found in cardiac fibroblast to be critical for cardiac fibrosis and data that most cardiac CSTF2 is expressed in cardiac fibroblast, inhibition of CSTF2 in cardiac fibroblast is tested for sufficiency to alter cardiac fibrosis. Recently, genes were delivered specifically into cardiomyocytes using troponin T (TnT) promoter in an AAV9 construct and a similar strategy is used again to deliver CSTF2 shRNA into cardiac fibroblasts using TCF21 promoter in an AAV9 construct. There are 2 main mouse groups: 1) control mice and 2) HF mice. For fibroblast-specific expression, there are 2 treatments: 1) AAV9-hTCF21-GFP (control ShRNA) and 2) AAV9-hTCF21-CSTF2 ShRNA-GFP. This makes a total of 4 mouse groups. The AAV9-hTCF21-GFP has been demonstrated to show selective expression in cardiac fibroblasts in wild type C57BL/6J male mice. Hence, human TCF21 promoter was used to generate a cardiac fibroblast specific AAV9. Thus, AAV9-hTCF21-CSTF2 ShRNA-GFP was generated to target cardiac fibroblasts vitro VectorBuilder (Chicago, IL). A dose of 2×10¹⁰ viral genome (vg) dose of either AAV9-hTCF21-CSTF2 ShRNA-GFP or AAV9-hTCF21-GFP (control ShRNA) diluted to 150 μl volume in sterile PBS is administered via tail vein one week prior to inducing non-ischemic HF in the mice. Longitudinal echocardiographic monitoring of cardiac structure and function (LV mass, EF, and fractional shortening) at baseline and weekly thereafter for 4 weeks post-Ang II infusion. Following euthanasia, heart weight-to-body-weight ratios and heart-weight to tibia-length are recorded for all animals. Hearts are paraffin embedded with cardiac structure preserved to allow for histological analysis of cardiac hypertrophy by H&E staining for ventricular remodeling and direct measurement of myocyte size (WGA) and fibrosis (Masson Trichrome). In addition, MRI scans are performed to assess in vivo fibrosis in the heart. Frozen tissue from the central LV region are collected for molecular analysis-gene expression of molecular markers of fibroblast-derived CSTF2, SMA and various genes (see Table 1) altered in fibrosis are tested by qRT-PCR and protein expression by western blotting and immunohistochemistry. These protocols are used to monitor fibrosis and the relative timing of their onset in mice with decreased CSTF2 expression in cardiac fibroblasts.

RNA-Seq and 3′UTR Analysis from Cardiac Fibroblast.

The degree of cardiac fibrosis is correlated in these AAV9-hTCF21-CSTF2 shRNA-GFP mice with alterations in APA in fibrotic mediators and monitor corresponding decreases in protein levels. For this test, primary cardiac fibroblasts are isolated as previously described from AAV9-hTCF21-CSTF2 shRNA-GFP mice or AAV9-hTCF21-GFP-shRNA control mice subjected to HF, and are extracted from the total RNAs from these fibroblasts for PAC-Seq. Global APA changes are identified using the DaPars and poly Aminer algorithm and confirmed APA in fibrotic mediators using qRT-PCR to determine dPAS usage as described in FIG. 3. Western blot analysis reveal changes in protein levels. Further, pathway analysis and validation of alterations in ECM proteins are performed. Whole heart RNA and protein lysates from the mouse models are examined and used for immunohistochemistry to track these changes in the heart which are stained for CSTF2 levels and up-regulation of identified proteins in the fibrotic heart. Together, this data verifies that CSTF2 knockdown lengthen 3′UTR of profibrotic genes and decreases production of key pro-fibrotic (protein) mediators in the setting of cardiac fibrosis. Finally, the key findings made in this mouse model are compared to CSTF2 and APA mediated 3′UTR lengthening in cells and tissues from fibrotic HF patients. This enables assessments of the translatability of these findings and guide the selection of most relevant pathways affected by CSTF2 down regulation and APA in cardiac fibrosis.

Cardiac fibroblast as previously described from naive AAV9-hTCF21-CSTF2 shRNA-GFP, AAV9-hTCF21-GFP control shRNA and wild type C57/BL6 (control) mice (n=10/group) and live cell proliferation assessed using xCELLigence, migration capacity using the scratch assay and cellular stiffness using a collagen gel contraction assays and Atomic Force Microscope (AFM) (Core facility at the Houston Methodist Research Institute). Further analysis of proliferation is performed by immunofluorescence dual staining the cells with Ki67 and Vimentin/Periostin (fibroblast proliferation) and Ki67 and α-SMA (myofibroblast proliferation). As well as morphology and cytokine expression, immunoblotting, Immunofluorescence, and qRT-PCR, are used to examine myofibroblast for SMA, profibrotic and ECM genes mentioned in Table 1.

The association between increased CstF64 and shortening of 3′UTR of profibrotic genes led to the consideration that CstF64 directly targets these genes in HF fibroblasts and promotes myofibroblast activation. The APA events and CstF64 dysregulation were investigated in a well-established model of in vitro fibrosis as well. The cardiac fibroblasts were isolated from the LV of a healthy mouse and treated with 10 ng/ml of TGFβ1 for 72 hours. The TGFβ1 treated mouse cardiac fibroblasts showed excessive deposition of COL1A compared to the controls. So, it was investigated whether this increased expression is a result of dysregulated APA in the treated fibroblasts. The immunofluorescence staining of TGFβ1 treated fibroblasts showed significant up-regulation of CstF64 compared to the untreated fibroblasts (FIG. 10A). Hence, in this model as well, changes in 3′UTR length were found in the COLLA gene correlating with its increased protein expression which was also in agreement with the up regulation of CstF64.

### Example 2: Cleavage Stimulating Factor 64 Depletion Mitigates Cardiac Fibrosis Through Alternative Polyadenylation

Alternative polyadenylation (APA) regulates gene expression by cleavage and addition of poly(A) sequence at different polyadenylation sites (PAS) in 3′UTR, thus, generating transcript isoforms with different lengths. Cleavage stimulating factor 64 (CstF64) is an APA regulator which plays a role in PAS selection and determines the length of 3′UTR. CstF64 favors the use of proximal PAS, resulting in 3′UTR shortening, which enhances the protein expression by increasing the stability of the target genes. This example investigates the role of CstF64 in cardiac fibrosis, a key event leading to heart failure (HF). It was determined that the expression of CstF64, key profibrotic genes, and their 3′UTR changes by calculating distal PAS (dPAS) usage in left ventricular (LV) tissues and cardiac fibroblasts from HF patients. CstF64 was upregulated in HF LV tissues and cardiac fibroblasts along with increased deposition of fibrosis genes such as COL1A and FN1 and significant shortening in their 3′UTR. In addition, HF cardiac fibroblasts showed increased transforming growth factor receptor β1 (TGFβR1) expression consistent with significant shortening in TGFβR1. Upon knockdown of CstF64 from HF fibroblasts, downregulation in pro-fibrotic genes corresponding to lengthening in their 3′UTR was observed. This finding shows an important role of CstF64 in myofibroblast activation and promotion of cardiac fibrosis during HF through APA. Therefore, targeting CstF64 mediated RNA processing approach in human HF provides a new therapeutic treatment strategy for limiting fibrotic remodeling.

Alternative polyadenylation (APA) regulates gene expression post-transcriptionally and produces different lengths of 3′ untranslated regions (3′UTR) of messenger RNA (mRNA), as well as an altered reading frame. APA has a major impact on diverse cell processes that include mRNA metabolism, protein localization, differentiation, development, senescence, and cell proliferation. Mammals have around 70% of their mRNA-encoding genes with more than one polyadenylation site (PAS), most of which are found in 3′UTR regions. The differential use of proximal or distal PAS can result in APA isoforms with shorter or longer transcriptions or 3′UTRs. This can affect the stability of mRNAs in various ways, such as altering the availability of microRNA (miRNA) binding sites, RNA binding protein (RBP) sites, and lncRNA binding sites, which are usually found at the 3′UTR; or altering the repression of mRNA by AU-rich elements (ARE). As a result, mRNAs with shorter 3′UTRs are more stable since they escape regulatory mechanisms and tend to be overexpressed. Similarly, mRNAs with lengthened 3′UTRs are typically under expressed.

The choice of PAS is determined by the interaction between cis-elements in pre-mRNA and existing levels of trans-acting protein complexes involved in cleavage and polyadenylation. There are nearly 20 core trans-acting proteins which include four protein complexes-cleavage and polyadenylation specificity factor (CPSF), cleavage stimulation factor (CstF), cleavage factor Im (CFIm), and cleavage factor IIm (CFIIm); and multiple other single proteins. The majority of these protein complexes favor the use of distal PAS, whereas CstF64, one of the three components of the CstF complex, has been demonstrated to use proximal PAS, resulting in transcripts with shortened 3′UTRs. Through its interaction with the conserved G/U-rich downstream sequence element (DSE), the CstF complex contributes to the selection of PAS. CstF64 is the RNA binding component of the CstF complex that has been associated with upregulation in many cancer types including those where 3′UTR shortening also occurred. As a result of the dysregulation of APA core proteins, global 3′UTR shortening events have been reported in several human diseases, including cardiovascular disease. However, studies of the role of APA and, specifically, CstF64 in heart failure (HF) and its involvement in the progression of cardiac fibrosis have not been previously investigated.

Cardiac fibrosis is the primary cause of HF, and it is often associated with other cardiovascular diseases. When cardiac fibroblasts are activated and differentiated into myofibroblasts, they deposit extracellular matrix proteins (ECM), especially collagen type I, which causes matrix stiffness, reduced tissue compliance, and impaired heart muscle function, all factors accelerating the progression of HF. Despite a significant increase in treatment options for other organs, there are no FDA-approved drugs to directly target cardiac fibrosis, indicating a crucial need for innovative therapeutic approaches. The molecular mechanisms driving the deposition of ECM proteins and the trigger of profibrotic genes are not completely understood, particularly with RNA regulation. Herein, the role of CstF64 in cardiac fibrosis is investigated using tissues and fibroblast cells derived from the left ventricle (LV) of HF patients collected during transplantation.

### Materials and Methods

### Human Heart Tissues Collection.

The tissue samples were obtained from the LV of human HF patients during transplantation at the DeBakey Heart & Vascular Center, Houston Methodist Hospital as described previously [11]. Control tissue samples were obtained from the LV of healthy donor patients that were not used for transplantation. The tissues were immediately flash frozen in liquid nitrogen and stored at −80 °C for protein and RNA isolation until use. Tissues were immediately fixed in 2% paraformaldehyde and the standard protocol was followed to embed them in paraffin. For cell culture, fresh tissues were immediately stored in cold phosphate-buffered saline (PBS). This protocol is approved by the Houston Methodist Hospital Institutional Review Board [IRB (2) 0511-0100].

### Cardiac Fibroblast Isolation and Culture.

Human LV tissues were collected in cold phosphate-buffered saline (PBS) and cut into small pieces about 1-2 millimeters in size. The LV tissues were digested with Liberase™ 0.56 U/ml (Roche, USA) in DMEM (Invitrogen, USA) at 37 °C for approximately 1 hour. The cell suspension was washed with DMEM 4-5 times and seeded into 6-well plates in a Fibroblast Growth Medium-3 (FGM-3) (Promocell, USA). Human cardiac ventricular fibroblast (Promocell, USA) derived from healthy hearts was cultured in FGM-3 and used as a control.

### Cell Transfection.

Human cardiac fibroblasts derived from the LV of the HF patients were grown in antibiotic-free media, transfected with 50 ng/ml siRNA (Santa Cruz Biotechnology, USA) using Lipofectamine RNAiMAX (Thermo Fisher Scientific, USA). Seventy-two hours post-transfection, cells were harvested for western blot, qRT-PCR, or fixed for immunofluorescence staining.

### Western Blot Analysis.

Cardiac tissues and cultured cardiac fibroblasts were lysed in RIPA buffer (Boston BioProducts, USA) with protease inhibitor (Thermo Scientific, USA) and phosphatase inhibitor (Thermo Scientific, USA). The total protein concentration in the samples was measured by the bicinchoninic acid method (Thermofisher Scientific, USA). Equal amounts of proteins were resolved by electrophoresis and transferred to PVDF membrane. The blots were blocked with a 1% casein blocker (Bio-Rad, USA) and incubated with primary antibodies following incubation with horseradish peroxidase conjugated secondary antibodies (Santa Cruz Biotechnology, USA). Clarity™ western ECL substrate (Bio-Rad, USA) was used to detect the bands. The photographs of stained immunoblots were taken with a BIO-RAD ChemiDoc imaging system (Bio-Rad, USA).

### Immunofluorescence Staining and Quantification.

Control and HF fibroblasts were grown to 70-80% confluency and fixed with 4% formaldehyde for 20 minutes at room temperature. Cells were stained with collagen type 1 alpha (COL1A), transforming growth factor-beta 1 (TGF-β1), fibronectin 1 (FN1), CstF64 (Proteintech, USA), and alpha-smooth muscle actin (α-SMA) (Sigma Aldrich, USA) antibodies. Images were acquired using EVOS™ M5000 microscope (Thermofisher Scientific, USA). The area stained by each marker was quantified using ImageJ (NIH) software following guidelines from NIH.

### Fibrosis Assessment.

Tissues sections from LV of HF and control patients were stained using a Masson's trichrome kit (Sigma-Aldrich, USA) following manufacturer's guidelines and images were taken using an Olympus AX70 microscope (Olympus, Japan). Fibrosis was quantified using the methods described previously.

RNA Isolation and RT-qPCR for dPAS Usage.

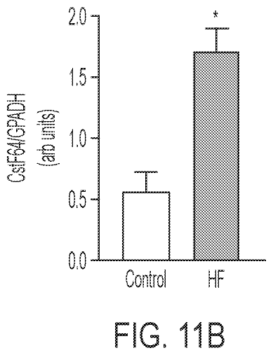

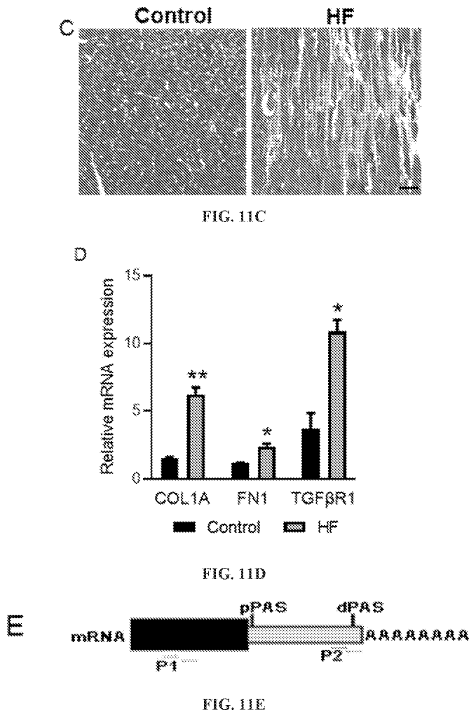

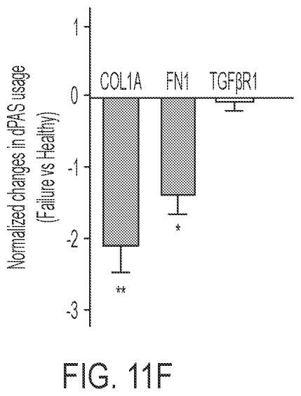

LV tissues and cultured cardiac fibroblasts were homogenized in QIAzol Lysis Reagent (Qiagen, USA) and isolated using the RNeasy Mini Kit (Qiagen, USA). cDNA was constructed using qScript® cDNA SuperMix (Quantabio, USA). Real-time PCR was performed using CFX96™ Real Time System (Bio-Rad, USA). Two pairs of primers were designed for each gene to calculate dPAS usage. The first pair was designed to target an open reading frame representing the total transcripts and the second pair was designed to target a region slightly proximal of distal PAS, which represented the mRNAs with longer 3′UTR (FIG. 11E). The dPAS usage was calculated as ΔCT=CT_{distal}−CTₜₒₜₐₗ. Data were presented as differences normalized to control by calculating ΔΔCT=ΔCT_{average target}−ΔCT_{average} of control. A negative ΔΔCT indicates 3′UTR shortening, and a positive value indicates 3′UTR lengthening compared to control.

### Statistical Analyses

Results are presented as mean±standard error mean (SEM). Unpaired Student's t-test was performed to test statistical differences between two groups. All the statistical analyses were carried out using the Graph Pad Prism software V9.0 (Graph Pad Software Inc., USA).

### Results

### LV of HF Patients Show Increased Expression of CstF64.

The HF LV tissues showed significantly increased expression of CstF64 compared to the healthy controls by western blot (FIGS. 11A and 11B). To assess the fibrosis state in the corresponding HF tissues, Masson's trichome staining was performed, which showed significantly increased (29.38±1.77%, ***p<0.001, n=3) fibrosis compared to the healthy controls (FIG. 11C). Similarly, the expression of key profibrotic genes, such as COL1A, FN1, and TGFβR1, were also significantly increased in the HF (FIG. 11D). So, it was asked if the difference in fibrosis was because of APA events by the promotion of 3′UTR shortening in profibrotic genes. To find if COL1A, FN1, and TGFβR1 are regulated by APA, RT-qPCR was used to calculate the percentage of mRNA that are using the distal Poly-A site (dPAS). Using this method, COL1A and FN1 had a greater number of shortened transcripts in HF compared to the healthy controls (FIG. 11F). The shortening in 3′UTR allows escaping from regulatory RNAs and proteins hence, increasing the protein expression. Here, the fibrosis state in HF was also correlated with the increase in the number of 3′UTR shortened transcripts of fibrosis genes.

Isolated Cardiac Fibroblasts from HF Patients Show Dysregulation of CstF64 and 3′UTR Shortening of Key Profibrotic Genes.

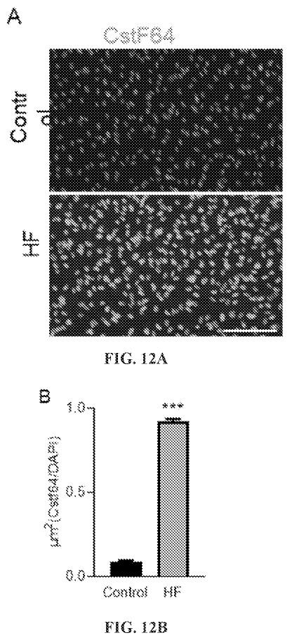

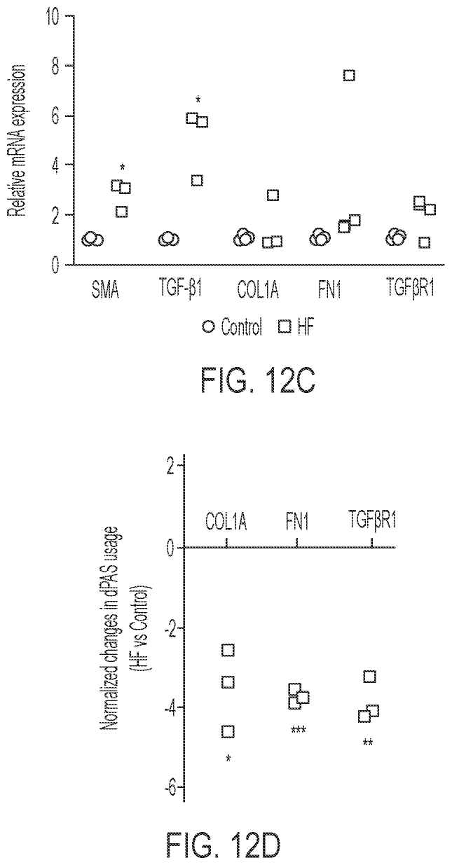

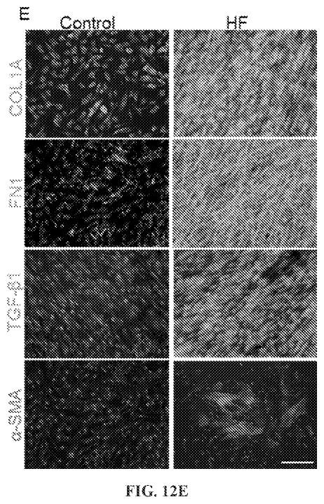

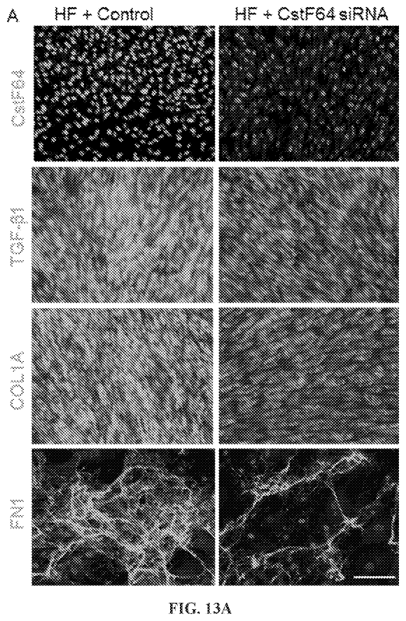

The immunofluorescence staining showed a significantly higher expression of CstF64 in cardiac fibroblasts from HF patients than the controls (FIGS. 12A and 12B). Using RT-qPCR, the transcript levels of some of the key profibrotic markers were investigated, including α-SMA, the marker of myofibroblast differentiation and activation. The transcript levels of α-SMA and TGF-β1 were significantly higher in cardiac fibroblasts from HF patients. Not all the HF cardiac fibroblasts showed significant differences in COL1A, FN1, and TGFβR1 expression compared to the healthy controls (FIG. 12C). On the other hand, the investigation of dPAS usage showed that there is a significant 3′UTR shortening of COL1A, FN1, TGFβR1 in cardiac fibroblasts from HF compared to healthy controls (FIG. 12D). Then, the protein expression of COLLA and FN1 along with other key genes in fibrosis, TGF-β1, and α-SMA, was investigated in the HF fibroblasts to relate them to 3′UTR shortening in these genes. The immunofluorescence staining of HF fibroblasts showed significantly increased deposition of ECM genes such as COL1A and FN1 compared to the healthy controls (FIG. 12E). In addition, a significant number of activated myofibroblasts marked by α-SMA and upregulated TGF-β1 expression were seen in HF fibroblasts compared to healthy controls (FIG. 12E). This confirmed the presence of α-SMA positive-myofibroblasts in HF, which were excessively depositing ECM proteins such as COL1A and FN1 as well as resulting in increased TGF-β expression. These data showed that irrespective of the total transcript levels in HF and control fibroblasts, 3′UTR shortening is sufficient to enhance the protein expression of those genes. Hence, these results demonstrated dysregulation of one of the key machinery of APA associated with an increase in 3′UTR shortening and protein levels of profibrotic markers.

Depletion of CstF64 from HF Fibroblasts Downregulates Profibrotic Genes.

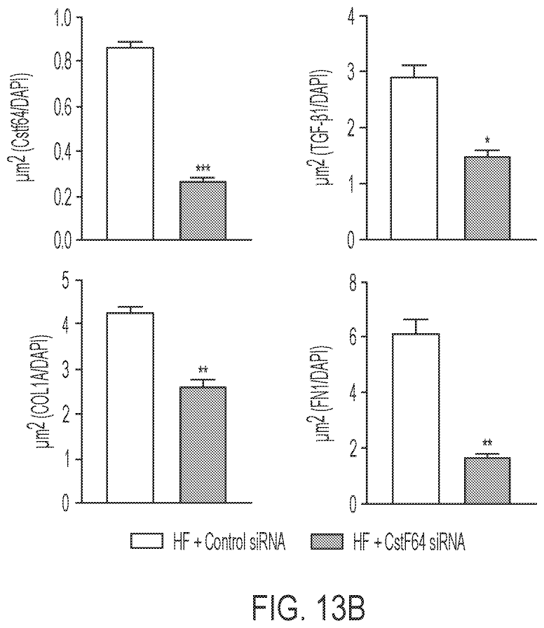

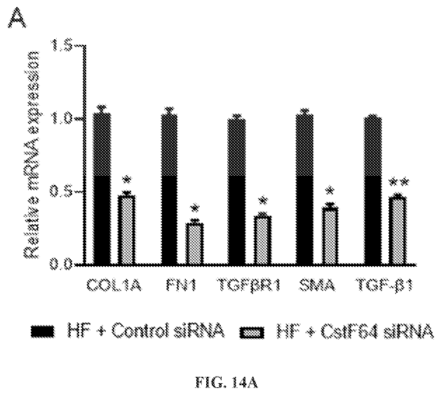

CstF64 is upregulated in HF fibroblasts and these fibroblasts also show 3′UTR shortening in some of the profibrotic genes that correlate with the protein expression. The association between increased CstF64 and shortening of 3′UTR of profibrotic genes lead to determining whether CstF64 directly targets these genes in HF fibroblasts and promotes myofibroblast activation. Therefore, the expression of CstF64 was knocked down in the HF fibroblasts using siRNA. The immunofluorescence staining of the HF fibroblasts transfected with CstF64 siRNA showed efficient knockdown of CstF64 after 72 hours compared to cells transfected with control siRNA (FIGS. 13A and 13B). With the efficient knockdown of CstF64, a decrease was seen in the expression of some of the key ECM genes such as FN1 and COL1A; and TGF-β1, a key cytokine implicated in fibrosis (FIGS. 13A and 13B).

### CstF64 Knockdown Changes the 3′UTR Length of Profibrotic Genes in HF Fibroblast.

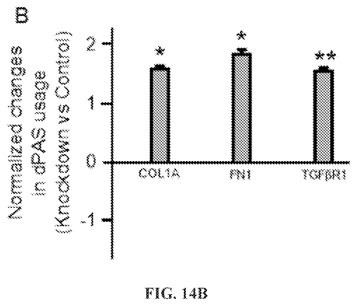

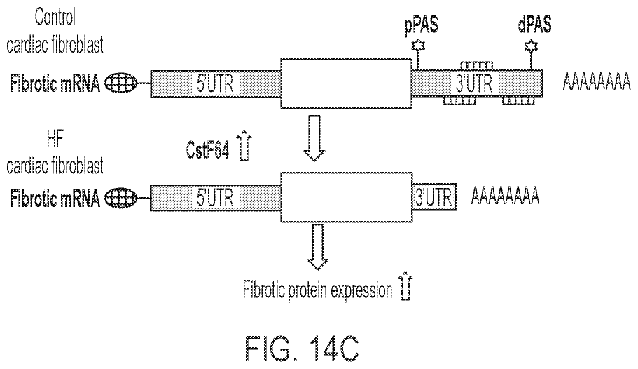

Next, the transcript levels of fibrosis genes were investigated after the knockdown by using RT-qPCR. The key fibrosis associated genes such as COL1A, FN1, and α-SMA, including the major cytokine TGF-β1 and its receptor TGFβR1, were significantly downregulated by at least 2-fold after the knockdown of CstF64 (FIG. 14A). Lastly, it was determined whether the knockdown of CstF64 changed the 3′UTR length of these target genes. RT-qPCR was performed to check the dPAS usage, and the data showed that the usage in dPAS increased after the knockdown of CstF64 (FIG. 14B). Compared to the control siRNA, there was an increase in the length of 3′UTR of COL1A, FN1, and TGFβR1 genes which also correlated with the downregulation of these genes observed at the protein level (FIG. 13A). Thus, CstF64 directly targets the 3′UTR region of COL1A, FN1, and TGFβR1 genes to regulate their protein expression in HF fibroblasts.

### Discussion

The present example has demonstrated that the APA machinery gene, CstF64, plays a significant role in the regulation of major profibrotic genes in LV cardiac tissues and isolated fibroblasts from patients with HF. A novel pro-fibrotic role for CstF64 is revealed by the shortening of 3′UTR length of profibrotic genes, which ultimately leads to the activation of myofibroblast differentiation. The expression of CstF64 was found to be upregulated in many cancer types which also exhibited global 3′UTR shortening. Upregulation of CstF64 has been shown to favor the use of weaker proximal PAS. In contrast, depletion of CSTF64 in Hela cells resulted in global 3′UTR lengthening. However, the role of CstF64 in 3′UTR shortening of fibrosis-associated genes in HF fibroblasts has not been investigated. First, a significant increase in CstF64 expression was shown in LV tissues from HF patients along with shortened 3′UTR in COL1A and FN1 genes and a consistent increase in collagen deposition in the LV failing tissues. This example confirmed an association between an increase in CstF64 and an increase in 3′UTR shortening events in the HF (FIG. 14C), similarly to what has been reported in various types of cancer.

The activation and differentiation of cardiac fibroblasts into ECM producing α-SMA-positive myofibroblasts is central to cardiac fibrosis. The expression of CstF64 was found to be higher in cardiac fibroblasts isolated from HF patients than in healthy controls. Aside from that, HF cardiac fibroblasts were α-SMA-positive, showed elevated levels of TGF-β signaling, and displayed an excess of genes known to be involved in the ECM, including COL1A and FN1. This example provides evidence for ECM genes including COL1A and FN1; and TGFβR1, a key receptor in TGF-β signaling, have shortened 3′UTR in HF fibroblasts. Although the total transcript levels of genes involved in fibrosis were not significantly higher in HF fibroblasts, the significantly shorter 3′UTRs of these genes explain the increase in the expression of those genes. Inhibitory miRNAs and RBPs negatively regulate transcript stability by binding mainly to 3′UTRs, thereby controlling protein expression. As a result of such expression of APA isoforms with shorter 3′UTR and without binding sites for miRNA regulation, nearly 10% of targeted miRNAs are altered, resulting in dysregulated protein synthesis. Similar mechanisms are thought to be involved in HF fibroblasts to alter genes associated with fibrosis expression by affecting the regulation by their target miRNAs. This showed APA events are also playing a role in myofibroblast activation and ECM deposition in addition to the canonical TGF-β signaling pathways.

Depletion of CSTF64 resulted in global 3′UTR lengthening and inhibition of the target genes. A significant finding here was that depleting CstF64 alone in cardiac fibroblasts isolated from HF patients was sufficient to induce 3′UTR lengthening in COL1A, FN1, and TGFβR1 genes; and reduce gene or protein expression of ECM genes such as COL1A and FN1. In addition, the knockdown of CstF64 in HF cardiac fibroblasts also showed downregulation of key marker of fibroblast to myofibroblast differentiation, α-SMA, and a key cytokine implicated in fibrosis-TGF-β1 and its receptor TGFβR1. These results showed that CstF64 depletion lengthened these key genes associated with fibrosis, proving that these genes are directly targeted by APA. Thus, the downregulation of profibrotic genes in HF cardiac fibroblasts induced by CstF64 knockdown shows that CstF64 may be a new therapeutic target for the treatment of cardiac fibrosis.

In conclusion, it is shown that the dysregulation of the key component of APA regulators, CstF64, in tissues and isolated fibroblasts from failing human hearts is correlated with 3′UTR shortening and increased expression of key profibrotic genes. The knockdown of CstF64 from the cardiac fibroblasts from HF patients mitigated the excess deposition of some ECM genes and downregulated the TGF-β1 cytokine and its receptor (components of a central profibrotic signaling pathway) consistently with lengthening of 3′UTR in those genes. This example provides new insights into the role of CstF64 via APA-dependent regulation of cardiac fibrosis.

### Example 3: CstF64 as a Biomarker

Animal models of heart failure have shown that CSTF64 is a proximal event leading to increase in profibrotic proteins and then leading to fibrosis. Currently, biomarkers in heart failure are surrogates of disease process, i.e., they are late markers of disease and mostly reflective of increase pressures in the heart cavities (atria and ventricles).

CSTF64 presents as an early marker of disease which is helpful in patients with heart failure with preserved ejection fraction since initiation of late treatment does not help in these patients. Serum levels of CSTF 64 can be measured in these patients and with sensitive PET imaging using a small molecule against CSTF64, it can be identified as an early marker of disease.

Expression of CstF64 protein was detected within human heart failure (HF) tissue by western blot. The HF human heart tissues showed significantly increased expression of CstF64 compared to the healthy controls by Western blot. In addition, HF mice hearts showed significantly increased expression of CstF64 by qPCR (8-fold increase, 7.825±0.600-fold CstF64/Actin, p<0.01) like that of human HF. Further, the expression of CstF64 was investigated in healthy, pre-LVAD (HF), and post-LVAD (recovered) hearts. The left ventricular tissue from a cohort of 5 HF patients' pre and post LVAD support was studied. Significantly increased CstF64 protein expression was observed in pre-LVAD HF tissues compared to healthy tissues (4.6-fold increase, 1.8±0.3116 densitometry CstF64/GAPDH, p<0.01). Interestingly, after LVAD support, CstF64 protein expression was significantly reduced compared to pre-LVAD support (2.09-fold decrease, −1.211±0.3094 densitometry CstF64/GAPDH, p<0.05).

In addition, a CSTF64 biomarker is not a surrogate since this measures the pathogenic step rather than manifestation.

### Example 4: CstF64 Depletion in Mice

### Methods

### Animals and Treatments

All animal studies were performed in accordance with approved protocols by the Houston Methodist Research Institute's Institutional Animal Care and Use Committee and carried out in accordance with the National Institutes of Health Guide for the Care and Use of Laboratory Animals.

Twelve-week-old C57BL6 mice (n=12) were randomly divided into three groups: Group1, Control (n=3); Group 2, Heart Failure with Control shRNA AAV (n=3) and Group 3, Heart Failure with CstF64 shRNA AAV (n=3) treatment. L-NAME (0.3 mg/mL with 1% NaCl) was administered in administered in drinking water to Group 2, 3 & 4, whereas Group1, control mice received regular drinking water. For Group 2, 3 and 4, 1 week after L-NAME treatment, the mice were anesthetized with inhaled isoflurane and subcutaneous osmotic pump (Alzet, no 1004) were implanted to deliver Angiotensin II (0.7 mg/kg per day) for 4 weeks, while the L-NAME water was continued. All mice were housed in micro isolation cages under 12 h: 12 h light/dark cycle and were fed a standard chow diet (Harlan Teklad, 2920). At the end of the 5-week heart failure induction, mice are assessed for cardiac function, and euthanized to collect heart tissue for further processing.

### Fibrosis Assessment

Tissues sections from three groups of mice were stained using a Masson's trichrome kit (Sigma-Aldrich, USA) following manufacturer's guidelines and images were taken using an Olympus AX70 microscope (Olympus, Japan). Fibrosis was quantified using the methods described previously.

### Hypertrophy Assessment

Paraffin-embedded tissue sections were stained with Hematoxylin and Eosin following standard operating procedures. Images were taken using an Olympus AX70 microscope (Olympus, Japan). Myocytes diameter was measured for 50 cardiac myocyte cells using Image J software following guidelines from NIH.

### Results

### CstF64 Depletion Attenuates the HF Phenotype in Mice

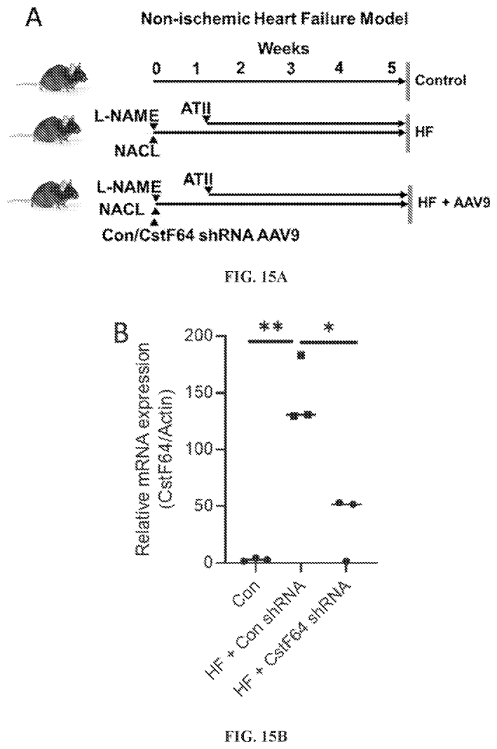

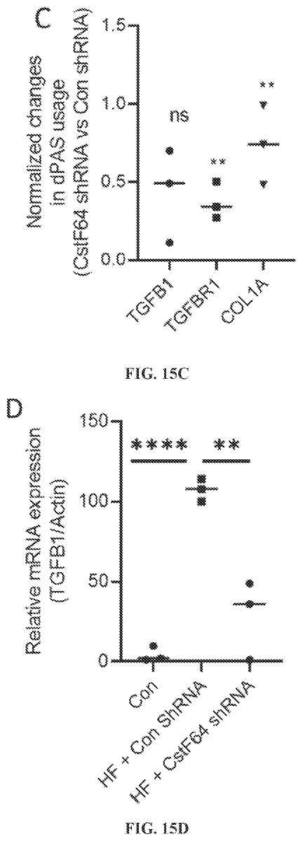

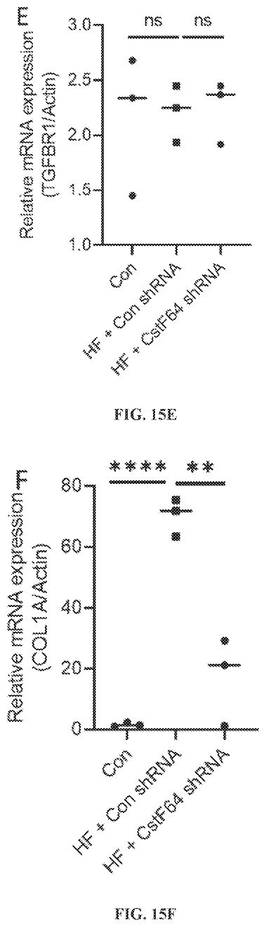

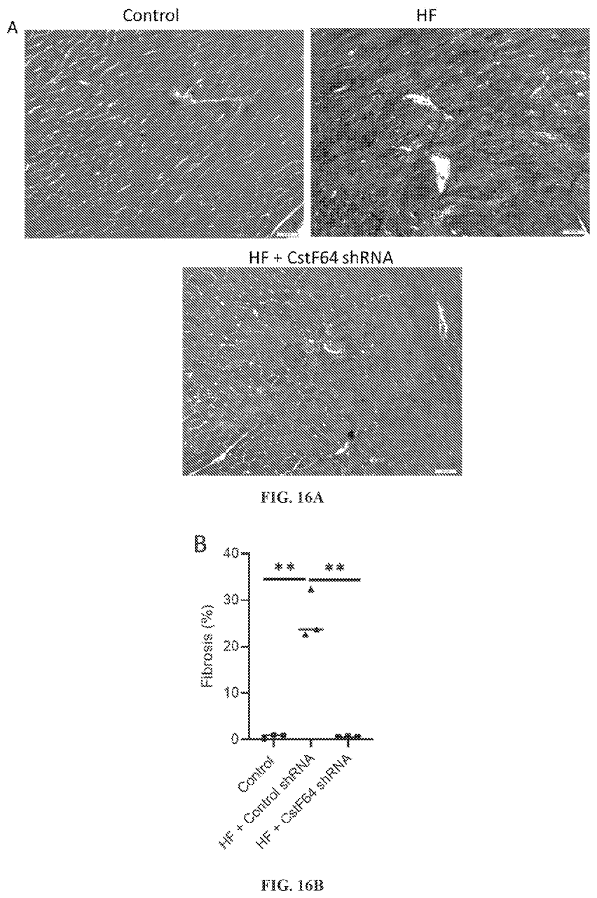

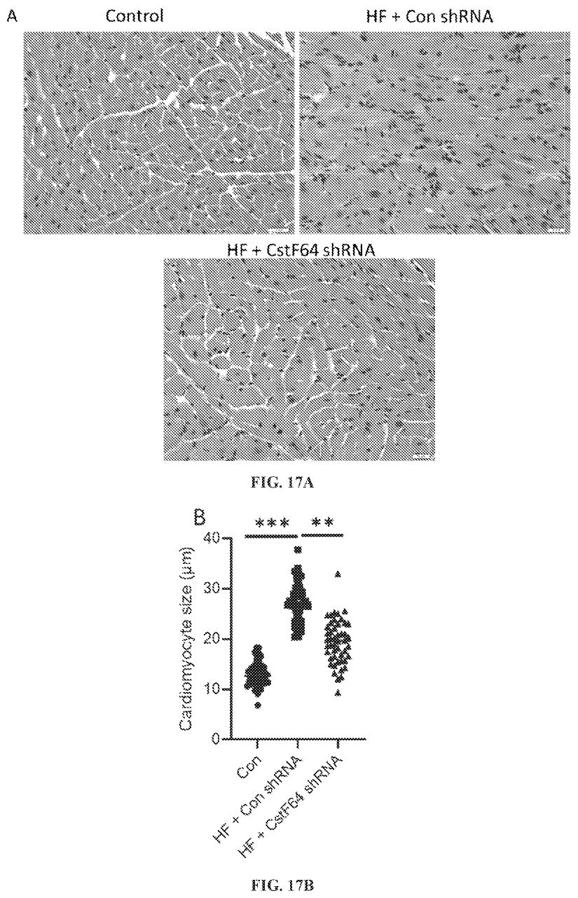

Since in vitro depletion of CstF64 in HF CFs was found to mitigate fibrosis and increase the 3′UTR length of profibrotic genes, its depletion in HF mice was tested for sufficiency in rescuing cardiac fibrosis. Thus, AAV9 viruses carrying CstF64 shRNA and GFP reporter under CMV promoter (AAV-CstF64 shRNA) or GFP reporter (AAV-Con shRNA, as control) (VectorBuilder-Chicago, IL) were generated. Herein, 1×10¹¹ viral genome (vg) dose of either AAV-CstF64 shRNA or AAV-Con shRNA diluted to 150 μl volume in sterile PBS was administered intraperitoneally one week prior to inducing HF in the mice (FIG. 15A). CstF64 was successfully depleted in HF mice compared to HF mice treated with AAV-Con shRNA (FIG. 15B). With the downregulation of CstF64, an increase in the 3′UTR length of profibrotic factors as well as downregulation in their gene expression except for TGFβR1 was observed (FIGS. 15C, 15D, 15E, and 15F). The HF mice subjected to CstF64 shRNA treatment showed significantly reduced cardiac fibrosis and hypertrophy (FIGS. 16 and 17). Thus, the results implicated in vivo role of CstF64 in attenuating cardiac fibrosis.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Fibrogenic Genes

| | |
|---|---|
| Fibrillar Collagens | I, III, V |
| Non-fibrillar collagens | IV, VI, VIII |
| Collagen synthesis and | TG2, LOX, MMP-1, -2, -8, -9, -13, |
| crosslinking | TIMP-1, -4, prolylhydroxylase |
| Matricellular protein | Thrombospondins, CCN2 |
| Glycoproteins | FN1, tenascin C, periostin |
| Proteoglycans | Heparan sulfate proteoglycan, decorin, |
| | fibromodulin, lumican, biglycan |
| Growth factors | PDGF-A, -B, TGFβ, BMP2 |
| Lectins | Galectin3 |
| Integrins | β1, β3, αv |

1. A method of treating or preventing cardiac fibrosis, comprising: administering to a subject in need thereof a therapeutically effective amount of a composition comprising an inhibitor of CstF64.

2. The method of claim 1, wherein the inhibitor of CstF64 is selected from a nucleic acid or a small molecule.

3. The method of claim 1, wherein the inhibitor of CstF64 comprises at least one siRNA.

4. The method of claim 1, wherein the inhibitor of CstF64 comprises one, two, or three siRNAs.

5. The method of claim 3, wherein the siRNAs comprises SEQ ID NO: 1, SEQ ID NO: 2, SEQ ID NO: 3, SEQ ID NO: 4, SEQ ID NO: 5, SEQ ID NO: 6, or any combination thereof.

6. The method of claim 3, wherein the siRNA comprises SEQ ID NO: 1.

7. The method of claim 3, wherein the siRNA comprises SEQ ID NO: 2.

8. The method of claim 3, wherein the siRNA comprises SEQ ID NO: 3.

9. The method of claim 3, wherein the siRNA comprises SEQ ID NO: 4.

10. The method of claim 3, wherein the siRNA comprises SEQ ID NO: 5.

11. The method of claim 3, wherein the siRNA comprises SEQ ID NO: 6.

12. The method of claim 1, wherein the inhibitor of CstF64 comprises an shRNA.

13. The method of claim 12, wherein the shRNA comprises SEQ ID NO: 7.

14. A method of treating or preventing heart failure, comprising:
administering to a subject in need thereof a therapeutically effective amount of a composition comprising an inhibitor of CstF64.

15.-29. (canceled)

30. A composition comprising an inhibitor cocktail and a pharmaceutically acceptable carrier, wherein the inhibitor cocktail comprises at least one inhibitor of CstF64 selected from SEQ ID NO: 1, SEQ ID NO: 2, SEQ ID NO: 3, SEQ ID NO: 4, SEQ ID NO: 5, SEQ ID NO: 6, or SEQ ID NO: 7.

31. The composition of claim 30, wherein the inhibitor cocktail comprises one, two, or three inhibitors of CstF64.

32. The composition of claim 30, wherein the inhibitor cocktail comprises an siRNA or an shRNA.

33. The composition of claim 30, wherein the inhibitor cocktail comprises SEQ ID NO: 1, SEQ ID NO: 2, SEQ ID NO: 3, SEQ ID NO: 4, SEQ ID NO: 5, SEQ ID NO: 6, or any combination thereof.

34.-39. (canceled)

40. The composition of claim 30,
wherein the inhibitor cocktail comprises SEQ ID NO: 7.
